(19) United States
(12) Patent Application Publication
(10) Pub. No.: US 2006/0161249 A1
(43) Pub. Date: Jul. 20, 2006

(54) Ring-shaped valve prosthesis attachment device

(76) Inventors: Fidel Realyvasquez, Palo Cedro, CA (US); Robb Moore, San Jose, CA (US); Jeff Abramson, San Jose, CA (US)

Correspondence Address: HELLER EHRMAN LLP, 275 MIDDLEFIELD ROAD, MENLO PARK, CA, 94025-3506, US

(21) Appl. No.: 11/286,270

(22) Filed: Nov. 22, 2005

Related U.S. Application Data
(60) Provisional application No. 60/629,984, filed on Nov. 22, 2004.
(60) Provisional application No. 60/709,185, filed on Aug. 17, 2005.

(51) Int. Cl. A61F 2/24

(52) U.S. Cl. 623/2.11; 623/2.38; 623/1.36

(57) ABSTRACT

A valve delivery device and method of use is provided. In one embodiment, a method for attaching a valve prosthesis to a target tissue includes providing a ring having a portion defining a groove and a plurality of fasteners coupled to the ring. A portion of the valve prosthesis is placed in the groove on the ring. The valve prosthesis portion in the groove is then secured to the ring. The ring is mounted on a delivery device and the delivery device is positioned so that the fasteners will engage the target tissue when the fasteners are deployed. A shaped plunger member may then deployed through an interior of the ring to swing said fasteners from a first position to a second tissue engagement position.

The present application claims the benefit of priority from U.S. Provisional Application 60/629,984 and 60/709,185 which are fully incorporated herein for all purposes.

### BACKGROUND OF THE INVENTION

1. Technical Field

The invention relates to apparatus and methods for prosthesis attachment and is especially useful in valve repair procedures.

2. Background Art

Essential to normal heart function are four heart valves, which allow blood to pass through the four chambers of the heart in one direction. The valves have either two or three cusps, flaps, or leaflets, which comprise fibrous tissue that attaches to the walls of the heart. The cusps open when the blood flow is flowing correctly and then close to form a tight seal to prevent backflow.

The four chambers are known as the right and left atria (upper chambers) and right and left ventricles (lower chambers). The four valves that control blood flow are known as the tricuspid, mitral, pulmonary, and aortic valves. In a normally functioning heart, the tricuspid valve allows one-way flow of deoxygenated blood from the right upper chamber (right atrium) to the right lower chamber (right ventricle). When the right ventricle contracts, the pulmonary valve allows one-way blood flow from the right ventricle to the pulmonary artery, which carries the deoxygenated blood to the lungs. The mitral valve, also a one-way valve, allows oxygenated blood, which has returned to the left upper chamber (left atrium), to flow to the left lower chamber (left ventricle). When the left ventricle contracts, the oxygenated blood is pumped through the aortic valve to the aorta.

Certain heart abnormalities result from heart valve defects, such as valvular insufficiency. Valve insufficiency is a common cardiac abnormality where the valve leaflets do not completely close. This allows regurgitation (i.e., backward leakage of blood at a heart valve). Such regurgitation requires the heart to work harder as it must pump both the regular volume of blood and the blood that has regurgitated. Obviously, if this insufficiency is not corrected, the added workload can eventually result in heart failure.

Another valve defect or disease, which typically occurs in the aortic valve is stenosis or calcification. This involves calcium buildup in the valve which impedes proper valve leaflet movement.

In the case of aortic valve insufficiency or stenosis, treatment typically involves removal of the leaflets and replacement with valve prosthesis. However, known procedures have involved generally complicated approaches that can result in the patent being on cardio-pulmonary bypass for an extended period of time.

Applicants believe that there remains a need for improved valvular repair apparatus and methods that use minimally invasive techniques and/or reduce time in surgery.

### SUMMARY OF THE INVENTION

The present invention involves valve repair apparatus and methods that overcome problems and disadvantages of the prior art. The present invention may facilitate the delivery and attachment of various prosthetic device into the body. The present invention may also reduce the amount time used to perform a delivery and attachment procedure.

In one aspect of the present invention, a method for attaching a valve prosthesis to a target tissue includes providing a ring having a portion defining a groove and a plurality of fasteners coupled to the ring. In this embodiment of the invention, a portion of the valve prosthesis is placed in the groove on the ring. The valve prosthesis portion in the groove is then secured to the ring. The ring is mounted on a delivery device and the delivery device is positioned so that the fasteners will engage the target tissue when the fasteners are deployed. A shaped plunger member may then deployed through an interior of the ring to swing the fasteners from a first position to a second tissue engagement position.

In another aspect of the present invention, a device for use with a valve prosthesis is provided. The device may include a ring and a first set of fasteners attached to the ring, wherein the fasteners are deformable to move from a first position to a second, tissue engagement position. The ring may define a groove for receiving at least one portion of the valve prosthesis.

In one embodiment of the present invention, a device for use with a valve prosthesis includes a ring and a first set of fasteners attached to the ring, wherein the fasteners are deformable to move from a first position to a second, tissue engagement position. The ring may defines a groove for receiving at least one portion of the valve prosthesis.

The present invention may also include a delivery system for use with the device wherein the system further comprises a shaped plunger member, wherein the shaped plunger member is movable along a longitudinal axis of the device and has a diameter sufficient to move the fasteners from a first position to a second, tissue engagement position. A support device may be expandable from a compressed configuration to an expanded configuration, the support device used to position the ring. The delivery system may include a support device that is expandable from a compressed configuration to an expanded configuration, the support device used to trap tissue between itself and the ring. The support device may be formed from a plurality of elongate support elements extending radially outward from a central disc, the support elements movable from a first position to a second, expanded position. The support device may be configured to be engaged by a shaped plunger member have a circumference sized to move support elements on the support device from a first position to second, expanded position. A shaped plunger member may be used that is sphere-shaped having a diameter sufficient to move the support element to the second position. The shaped plunger member may be mounted to shaft that is slidably mounted within a shaft coupled to the support device, the shape member movable relative to the support device. The shaped plunger member may be mounted to shaft that is slidably mounted over a shaft coupled to the support device, the shape member movable relative to the tissue connection device.

The fasteners may have a curved distal portion. The fasteners may have a sharpened distal tip. The fasteners have a blunt distal tip. They may be made of stainless steel. The fasteners in the first position may extend in part towards the inner circumference of the ring. The fasteners may have a first portion that is straight and a second portion that is curved. The fasteners may have a first portion extending in a first direction and a second portion extends in a second direction. The device may include a mesh or a polymer membrane coupled to the fasteners to minimize fluid leakage between the valve prosthesis and the target tissue once the valve prosthesis is delivered.

In another embodiment according to the present invention, a method for attaching a valve prosthesis to a target tissue. The method may include providing a ring having a portion defining a groove and a plurality of fasteners coupled to the ring; placing a portion of the valve prosthesis in the groove on the ring; securing the valve prosthesis portion in the groove to the ring; mounting the ring a delivery device; positioning the delivery device so that the fasteners will engage the target tissue when the fasteners are deployed; and advancing a shaped plunger member through an interior of the ring to swing the fasteners from a first position to a second tissue engagement position.

The method may include expanding a support device to an expanded configuration and moving the support device to engage a bottom surface of the target tissue. The method may include removing the delivery device and leaving the ring with the valve prosthetic attached to the target tissue. The target tissue may be an aortic valve annulus with valve leaflets removed.

The present invention may include a kit comprising a valve prosthesis delivery device; a ring having a plurality of fasteners; a valve prosthesis; instructions for use setting forth the method; and a container sized to house the valve prosthesis delivery device, the valve prosthesis, and the instructions for use.

Alternatively, the kit may include a ring having a plurality of fasteners; a valve prosthesis; instructions for use setting forth the method; and a container sized to house the valve prosthesis delivery device, the valve prosthesis, and the instructions for use.

In one embodiment, the present invention may also include a ring with a flexible annular ring. The ring may have a plurality of fasteners to secure the ring to tissue. Other embodiments may also have fasteners to secure a valve prosthesis to the ring.

In yet another embodiment of the present invention may include a deployment device that walks the circumference of the ring to provide radially outward force. The edge of the disc may be angled to provide outward and upward force.

The above is a brief description of some deficiencies in the prior art and advantages of the present invention. Other features, advantages, and embodiments of the invention will be apparent to those skilled in the art from the following description and accompanying drawings, wherein, for purposes of illustration only, specific forms of the invention are set forth in detail. A further understanding of the nature and advantages of the invention will become apparent by reference to the remaining portions of the specification and drawings.

A further understanding of the nature and advantages of the invention will become apparent by reference to the remaining portions of the specification and drawings.

### DESCRIPTION OF THE SPECIFIC EMBODIMENTS

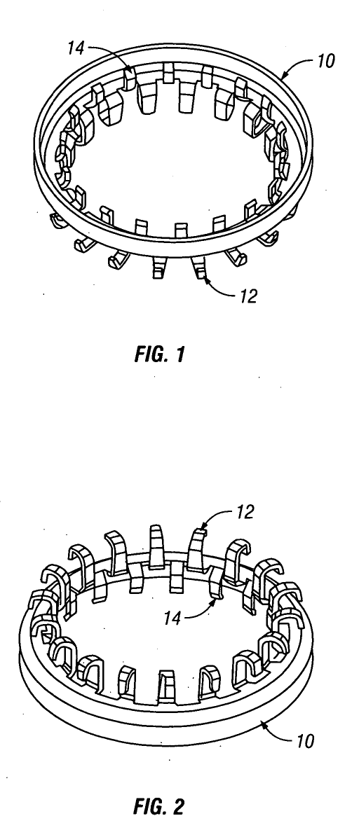

FIGS. 1 through 3 show one embodiment of a ring according to the present invention.

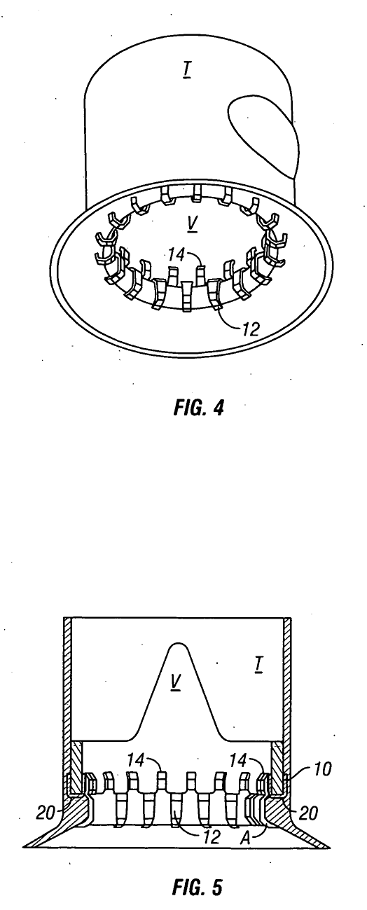

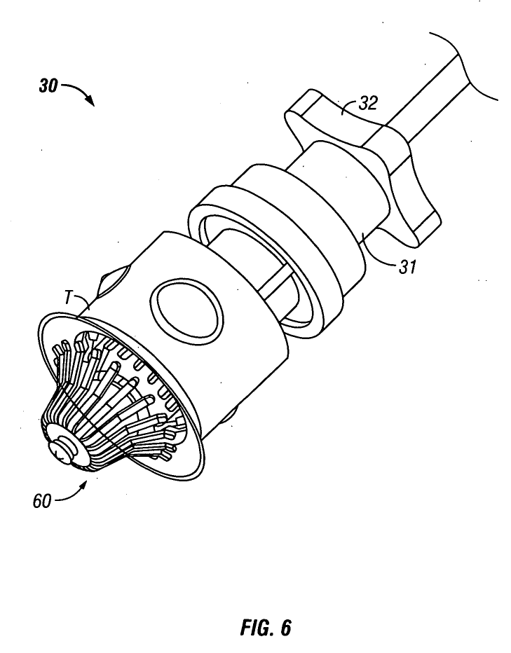

FIG. 4 through 5 are views of a ring and a valve prosthesis secured in place.

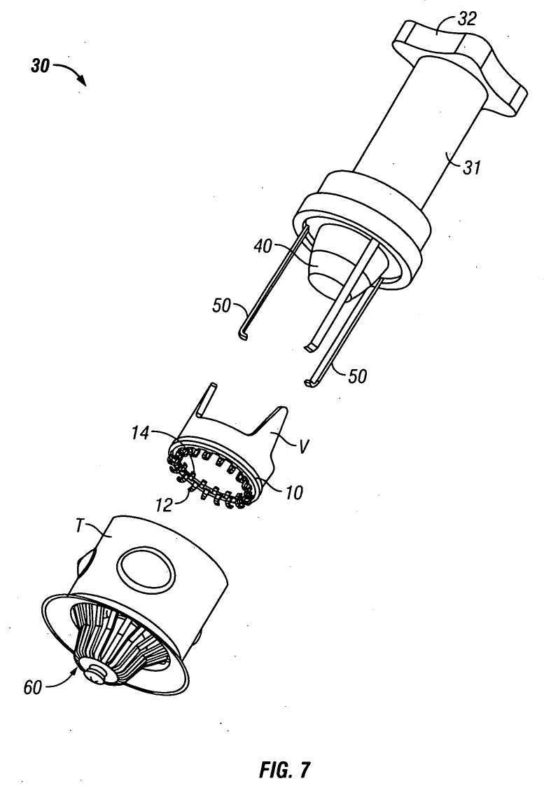

FIGS. 6 and 7 show a delivery device.

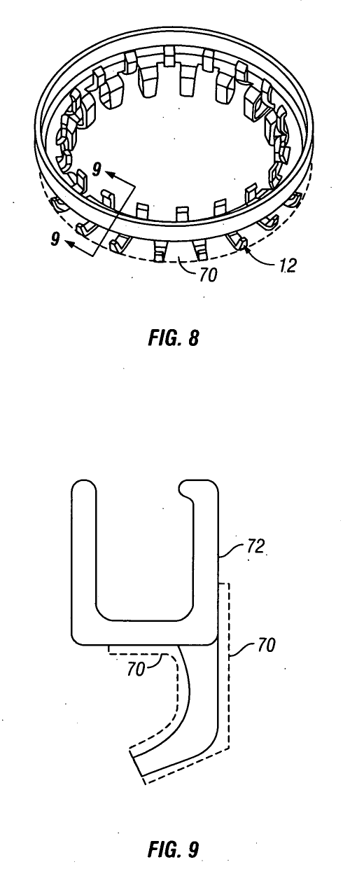

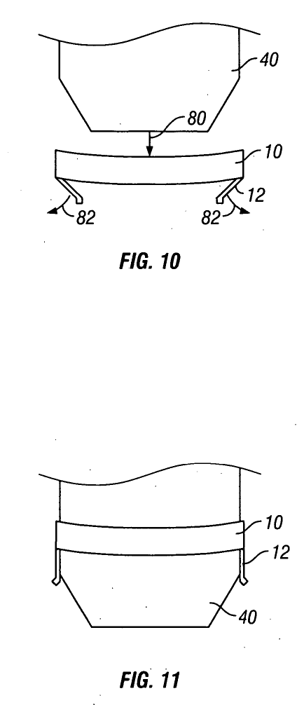

FIGS. 8 and 9 show fasteners on a ring FIGS. 10 through 12 show one method of deploying fasteners according to the present invention.

FIGS. 13 and 14 show side views of one embodiment of the invention.

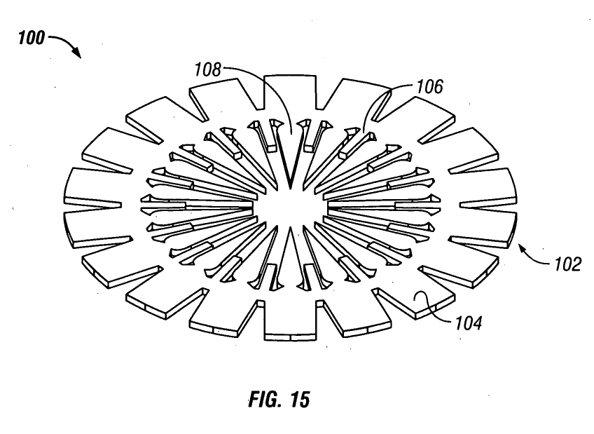

FIGS. 15 through 17 show perspective views of the present invention.

FIGS. 18 and 19 are views of a ring and a valve prosthesis secured in place.

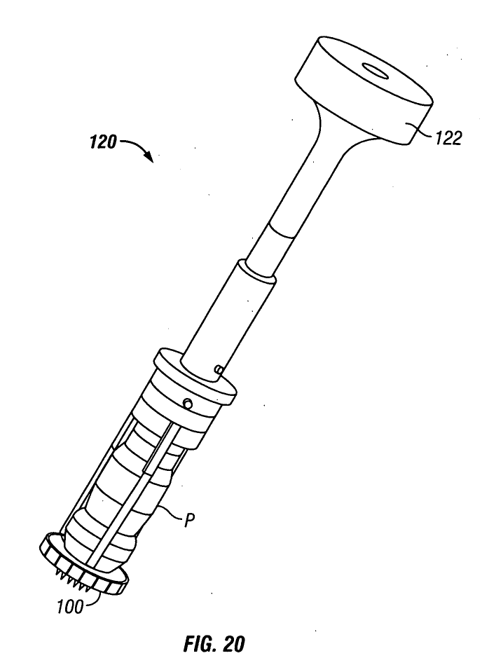

FIGS. 20 through 24 show one embodiment of a delivery device according to the present invention.

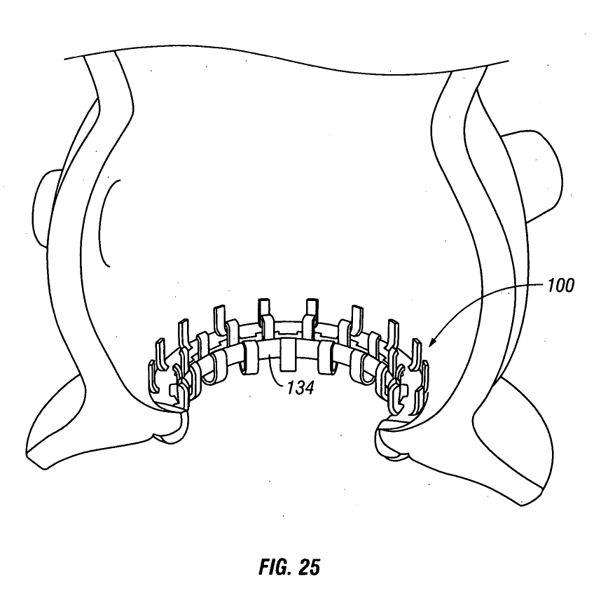

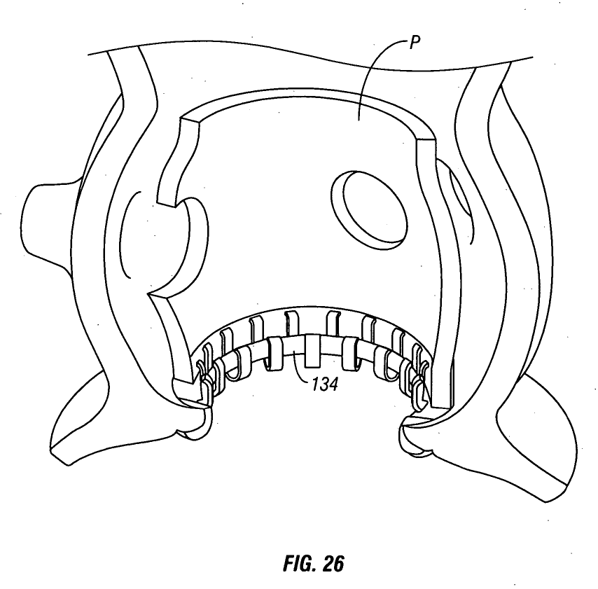

FIGS. 25 through 27 are views of a ring and a valve prosthesis secured in place.

FIGS. 28 through 33 show various embodiments of rings according to the present invention.

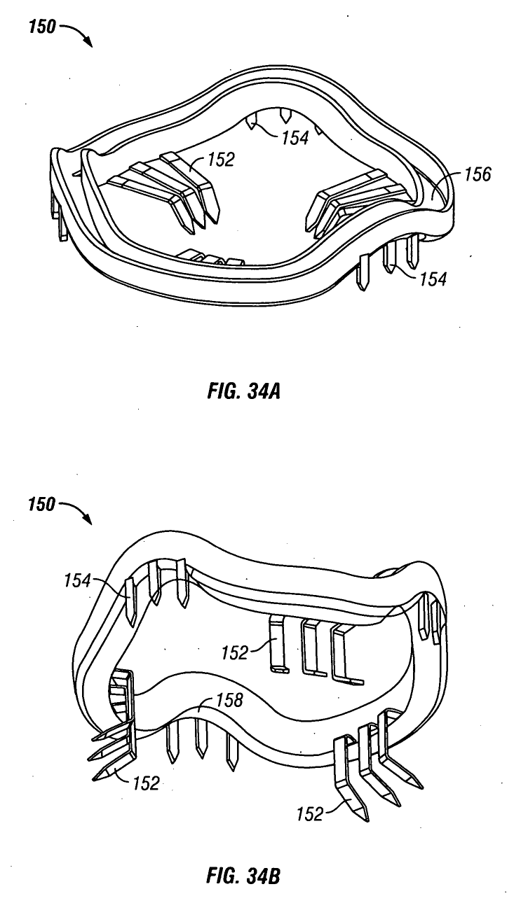

FIGS. 34 through 36 show a contoured ring.

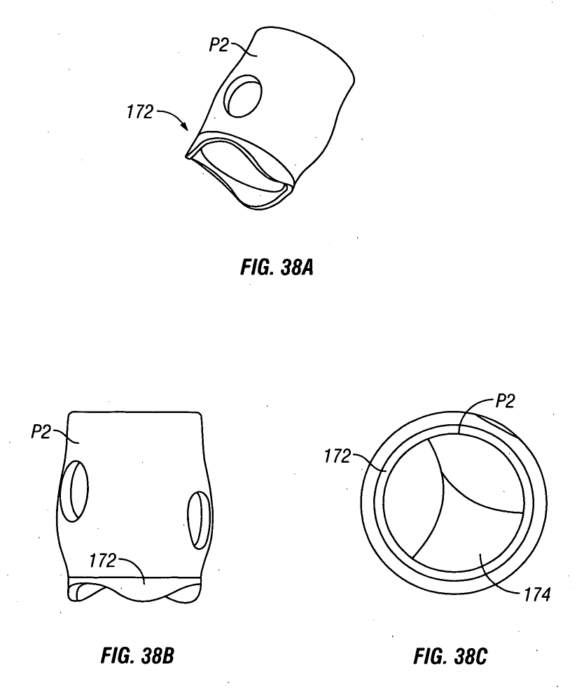

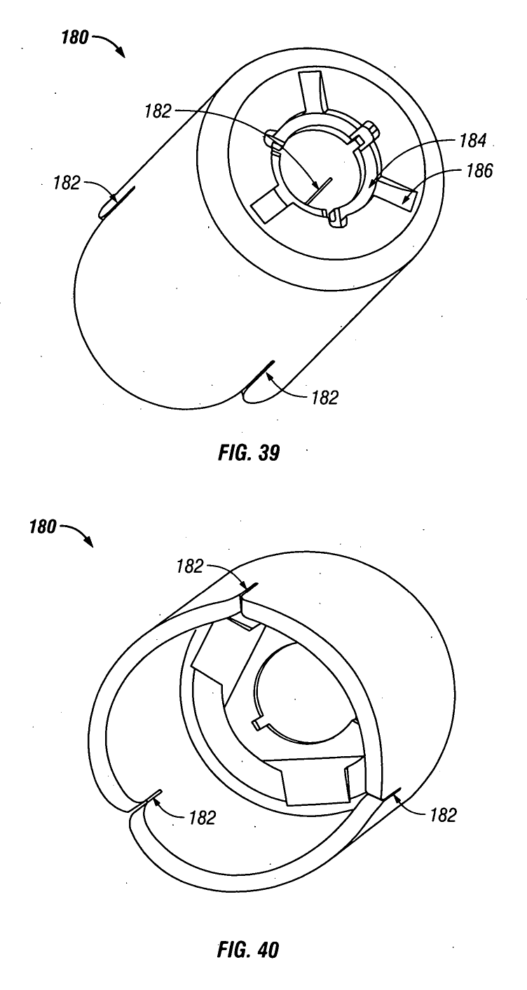

FIGS. 37 through 38 show a ring coupled to a valve.

FIGS. 39 through 48 show methods of mounting a ring and valve to a delivery device.

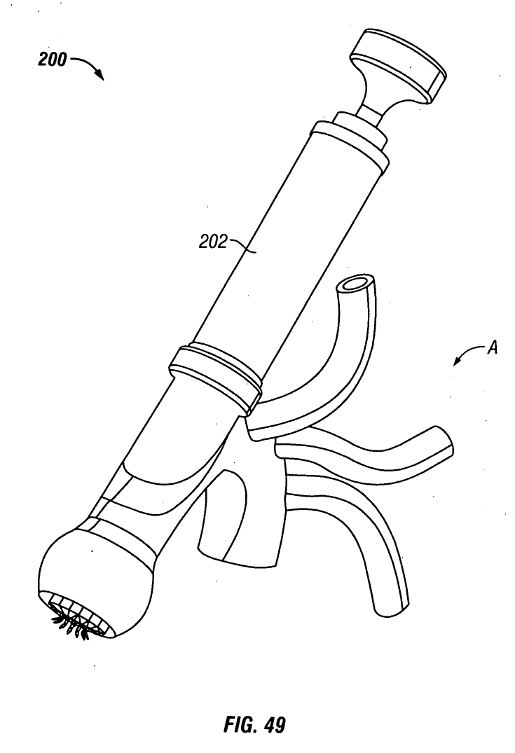

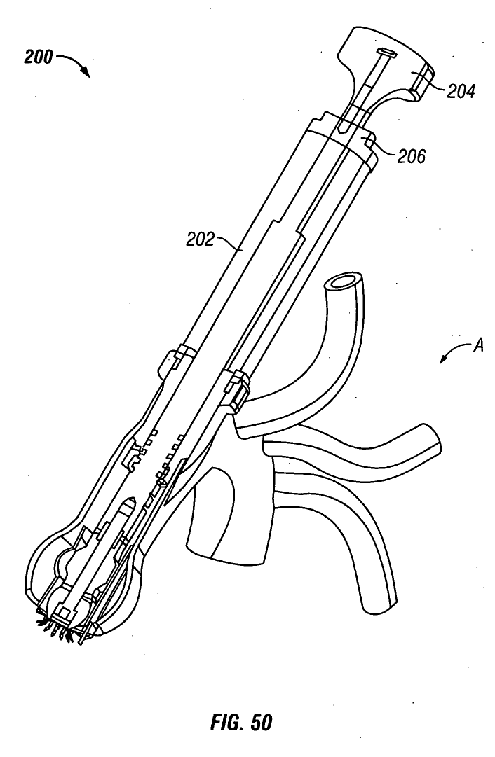

FIGS. 49 through 50 show a delivery of a ring to a target tissue.

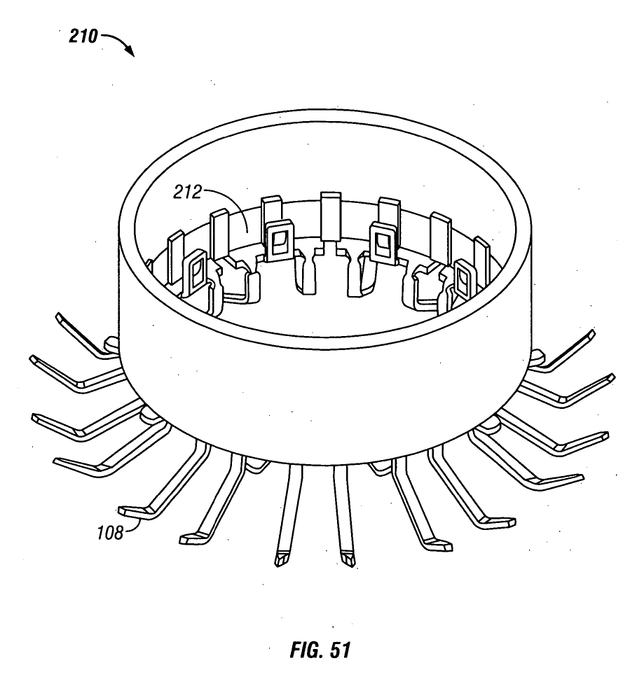

FIGS. 51 through 55 shows embodiments of a rings formed from individual segments.

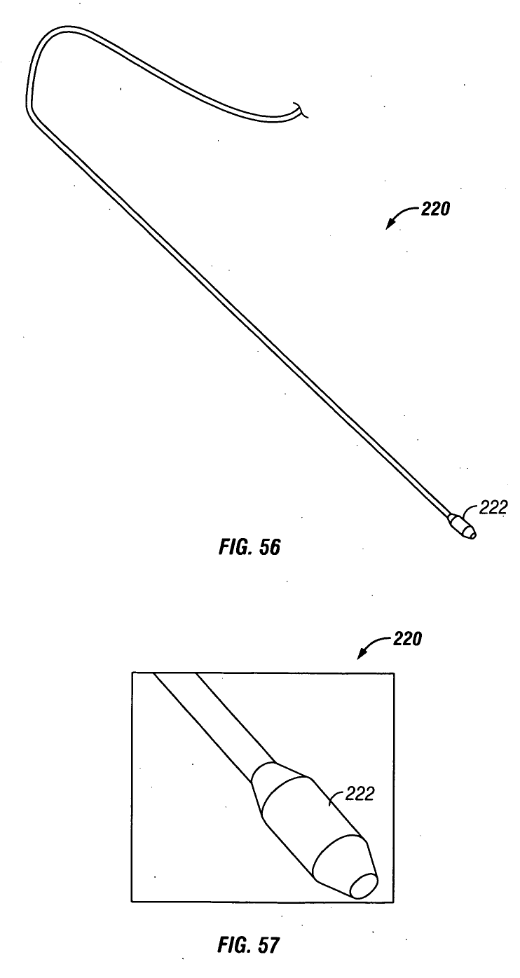

FIGS. 56 through 60 show embodiments of an imaging device for use with the present invention.

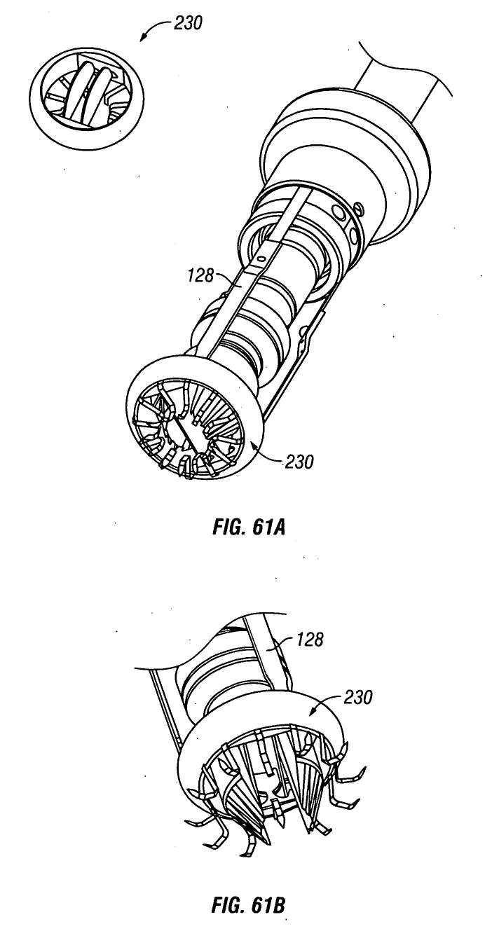

FIGS. 61 through 66 show embodiment of a ring for use with a mechanical prosthetic valve.

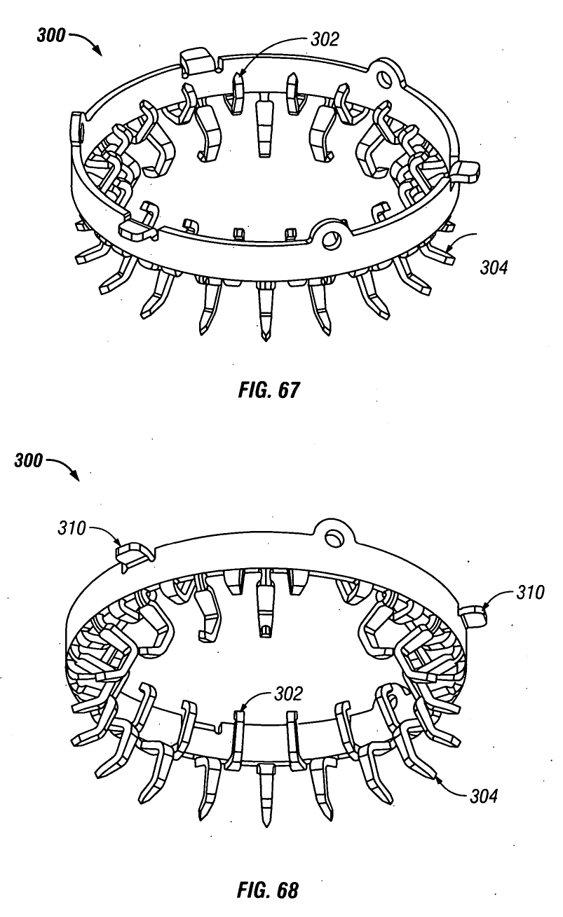

FIGS. 67 through 69 show embodiment of another embodiment of a ring according to the present invention.

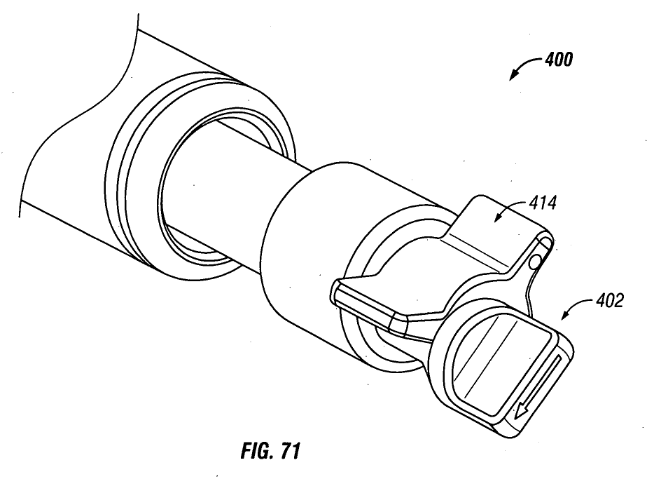

FIG. 70 shows a ring with a flexible annulus.

FIGS. 71 through 76 show another embodiment of a delivery device according to the present invention.

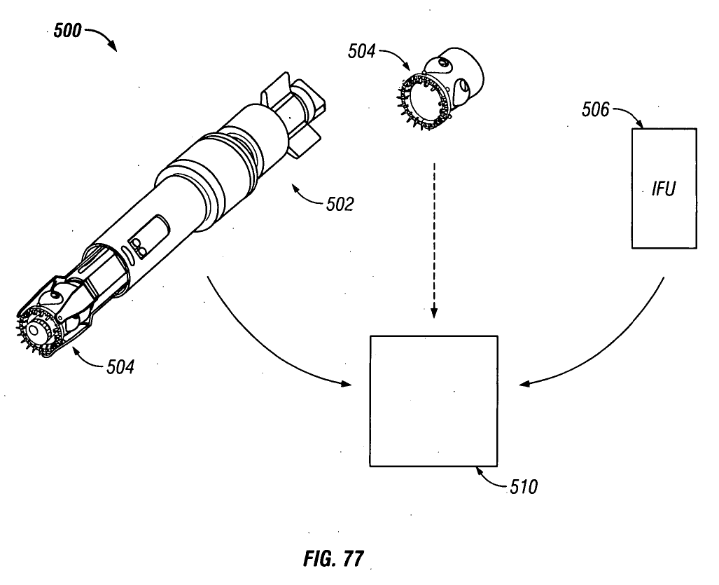

FIG. 77 shows one embodiment of a kit according to the present invention.

### DESCRIPTION OF THE SPECIFIC EMBODIMENTS

It is to be understood that both the foregoing general description and the following detailed description are exemplary and explanatory only and are not restrictive of the invention, as claimed. It may be noted that, as used in the specification and the appended claims, the singular forms “a”, “an” and “the” include plural referents unless the context clearly dictates otherwise. Thus, for example, reference to “a material” may include mixtures of materials, reference to “a chamber” may include multiple chambers, and the like. References cited herein are hereby incorporated by reference in their entirety, except to the extent that they conflict with teachings explicitly set forth in this specification.

In this specification and in the claims which follow, reference will be made to a number of terms which shall be defined to have the following meanings:

“Optional” or “optionally” means that the subsequently described circumstance may or may not occur, so that the description includes instances where the circumstance occurs and instances where it does not. For example, if a device optionally contains a feature for analyzing a blood sample, this means that the analysis feature may or may not be present, and, thus, the description includes structures wherein a device possesses the analysis feature and structures wherein the analysis feature is not present.

Referring now to FIGS. 1 and 2, one embodiment of the present invention will now be described. In this embodiment a ring 10 is provided with a first set of fasteners 12 integrated on to the ring. By way of example and not limitation, the fasteners 12 may be integrally formed with the ring and then laser cut or otherwise shaped into their final form and position. As seen in FIG. 1, the present embodiment may optionally include a second set of fasteners 14 which extend in a direction substantially opposite that of the first set of fasteners 12. As will be discussed later, this second set of fasteners 14 may be used to secure a prosthesis to the ring 10. By way of example and not limitation, the prosthesis may be a valve prosthesis or some other vascular prosthesis.

FIG. 2 shows another view of the device of FIG. 1 where the ring 10 is flipped vertically to show the fasteners 12 pointed upward. The embodiments of FIGS. 1 and 2 show the fasteners in a deployed configuration, where the fasteners 12 would have engaged a target tissue.

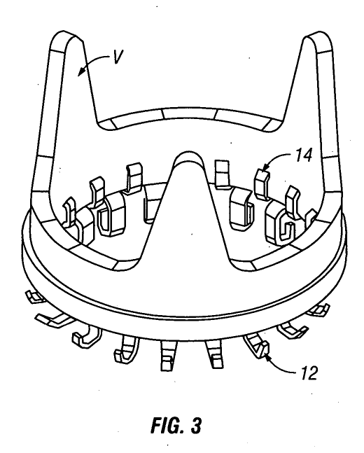

Referring now to FIG. 3, the prosthetic valve V is shown attached to the ring 10. The fasteners 14 may be used to attach the prosthetic valve V to the ring. In other embodiments, the prosthetic valve V may be formed directly on to the ring, removing the need for the second set of fasteners 14. In some embodiments, a mesh may be used to cover the gaps between the fasteners 12. This is more clearly shown in FIG. 8. Although not limited to the following, the mesh may be made of Dacron or polymeric material suitable for sealing. Some embodiments may use a membrane made of a polymer material. These materials may prevent fluid leakage between the fasteners when the valve V is deployed.

Referring now to FIGS. 4 and 5, the ring 10 is shown as mounted via fasteners 12 in a portion of aortic tissue T. FIG. 4 shows a perspective view. FIG. 5 shows a cross-sectional view of aortic tissue T. As seen more clearly in FIG. 5, the fasteners 12 will grip and/or penetrate target tissue at the aortic annulus A. FIG. 5 also more clearly shows a groove 20 defined by a portion of the ring 10 and the fasteners 14. This groove is sized to connect to the valve V. It should be understood, of course, that in some embodiments, the valve V may be integrally formed with the ring. By way of example and not limitation, the valve V may be molded or attached by adhesive or other material to the ring.

Again, the FIGS. 4 and 5 show fasteners 12 in a deployed configuration where the fasteners 12 have been formed and made to swing and hook to engage target tissue for securing the valve V in position. Prior to deployment, the fasteners 12 are in a more radially inward position once the fastener is positioned, they are urged outward through the use of a shaped plunger member 40 in FIG. 7 such that the tips of the fasteners can penetrate the valve tissue.

Referring now to FIGS. 6 and 7, perspective views are shown of a device 30 for deploying the ring 10 with the valve V. FIG. 6 shows a plunger 31 with a handle 32 pushed towards a distal end of the device to push the fasteners 12 to engage a target tissue at the annulus A. As seen in FIG. 6, the ring 10 may be keyed to align the fasteners 12 to swing in gaps between the fingers of tissue support device 60.

FIG. 7 more clearly shows how the fasteners 12 will be deployed. In this ring embodiment, a plunger 31 has a shaped plunger member 40 on a distal end. This shaped plunger member 40 will travel down the longitudinal axis of the device and enter the central opening of the valve V. As the shaper member 40 is deployed distally, it has a circumference sufficient to urge the fasteners 12 radially outward. Thus, the member 40 is used to swing out the fasteners to engage tissue. The device may use fasteners 12 made of materials such as but not limited to stainless steel. The fasteners 12 should go through a plastic deformation to grasp the tissue and remain in this new configuration. The tips of fasteners 12 may be sharp or blunt. As seen in FIG. 7, supports 50 may be used to hold the ring 10 in place while the shaped plunger member 40 traverses through the middle of the ring 10. In other embodiments, the support device or anvil 60 may also be used to engage the fasteners 12 and push them further in place by retracting on the fasteners.

FIG. 8 shows one embodiment of the present invention where mesh 70 or other material may be placed over and/or between fasteners 12. This may reduce the possibility of leakage once the ring 10 is coupled to the annulus A.

FIG. 9 shows that the mesh 70 may be placed in one embodiment on the inner circumference 72 or it maybe placed on an outward surface 74 to be positioned between the fastener 12 and the annulus A. Still other embodiments may have the mesh or material 70 on both the inner and outer surfaces.

Referring now to FIGS. 10 and 11, one method for deploying the fasteners 12 will be described. As seen in FIG. 10, the shaped plunger member 40 will be advanced as indicated by arrow 80 into the inner circumference defined by ring 10. As the shaped plunger member 40 advances, it will have a surface that will engage the plurality of fasteners 12. As the shaped plunger member 40 has a gradually increasing outer circumference, advancing the member 40 will swing the fasteners 12 outward as indicated by arrows 82.

FIG. 11 shows the shaped plunger member 40 having advanced to a position where the fasteners have moved to engage a target tissue and attach the ring 10 in position. At this point, the process may end and the shaped plunger 40 may be retracted and the entire device removed.

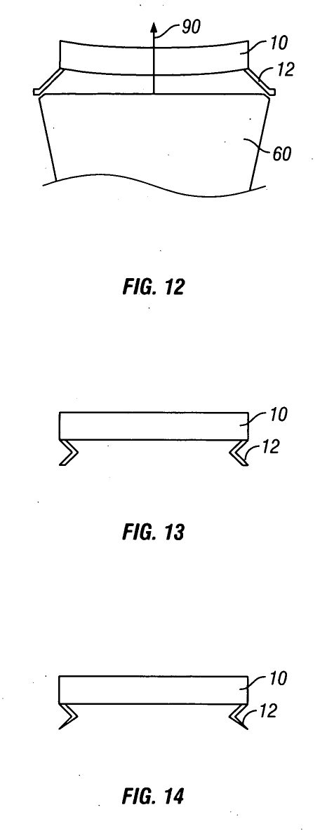

FIG. 12 shows a still further step where the support 60 may be expanded and then retracted as indicated by arrow 90 to drive the fasteners 12 further into the tissue. Some embodiments may expand the support 60 to have a larger circumference when it is in contact with fastener 12.

FIGS. 13 and 14 show that the fasteners 12 may also be configured to be more tissue penetrating and curved so that they will penetrating further into the tissue using just the shaped plunger 40.

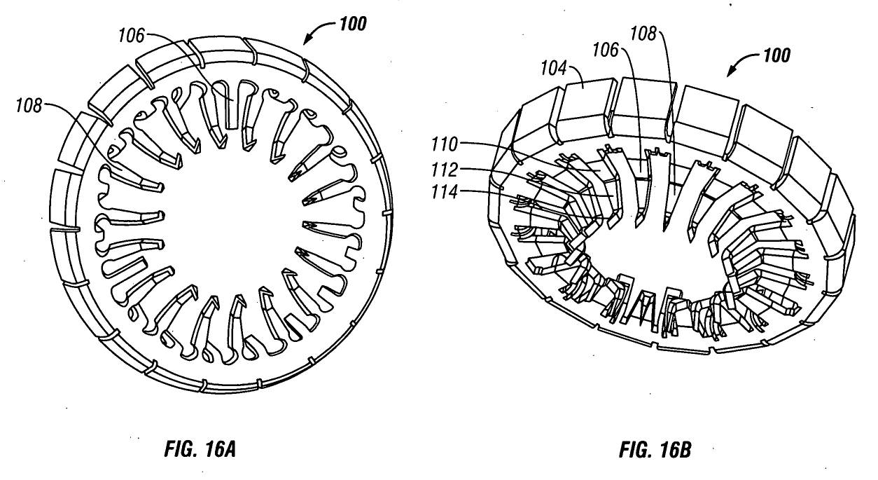

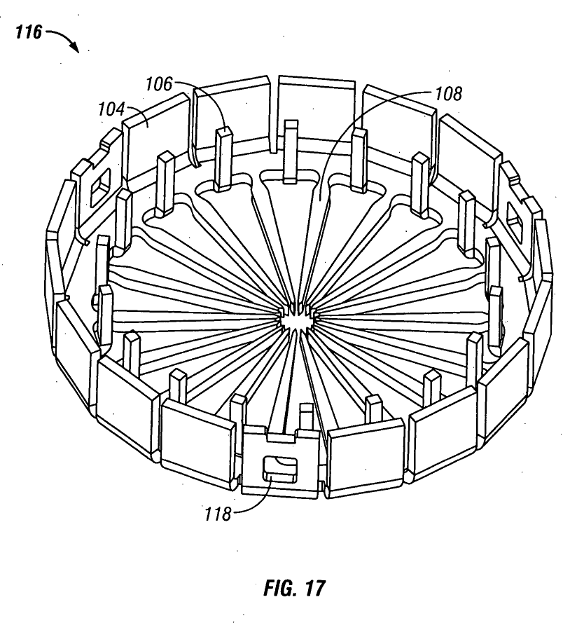

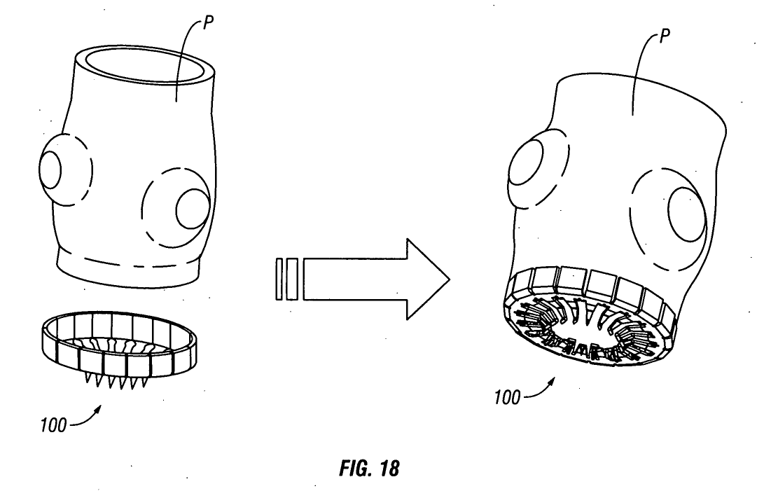

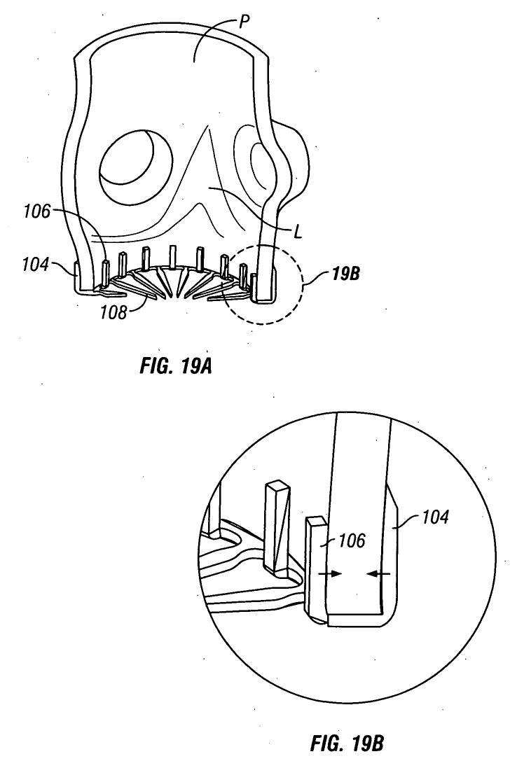

Referring now to FIGS. 15 through 17, a still further embodiment of the present invention will now be described. FIG. 15 shows a ring 100 in a planar configuration 102. The embodiment of FIG. 15 may be considered an intermediary. As a nonlimiting example, this planar configuration 102 may be laser cut, stamped, or otherwise cut and/or formed from a sheet of material. This planar configuration 102 is an intermediate form from which a ring 100 may be formed through bending and/or reshaping of the material. In the planar configuration 102, the outer peripheral wall is formed from a plurality of flanges or tabs 104 which may be shaped to bend upward (see FIG. 16A). A first set of tines 106 and a second set of tines 108 may be bent or shaped to become the fasteners used to secure the valve prosthesis (not shown) to the ring 100 and to attach the ring 100 to tissue, respectively. The tines 106 and 108 may have different shapes, different lengths, etc . . . to best achieve their respective objectives. In the present embodiment of FIG. 15, the tines 108 have a pointed tip and a longer length than tines 106 (about twice as long).

FIGS. 16A and 16B show bottom and top perspective views of the ring 100. FIG. 16A shows a bottom view with all of the tines 106 bent to engage the valve prosthesis (except for three of the tines). FIG. 16B shows a top view of the ring 100 with the tines 108 bent downward. Tines 106 are in their un-bent configuration in FIG. 16B. The tines 108 are shaped to have a horizontal portion 110, an angled portion 112, and downward portion 114. In one embodiment, the downward portion 114 may have a sharpened and/or pointed tip to facilitate tissue penetration.

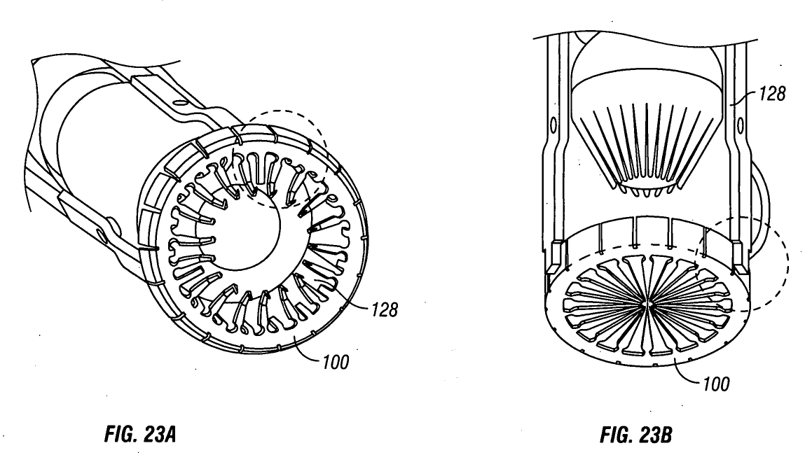

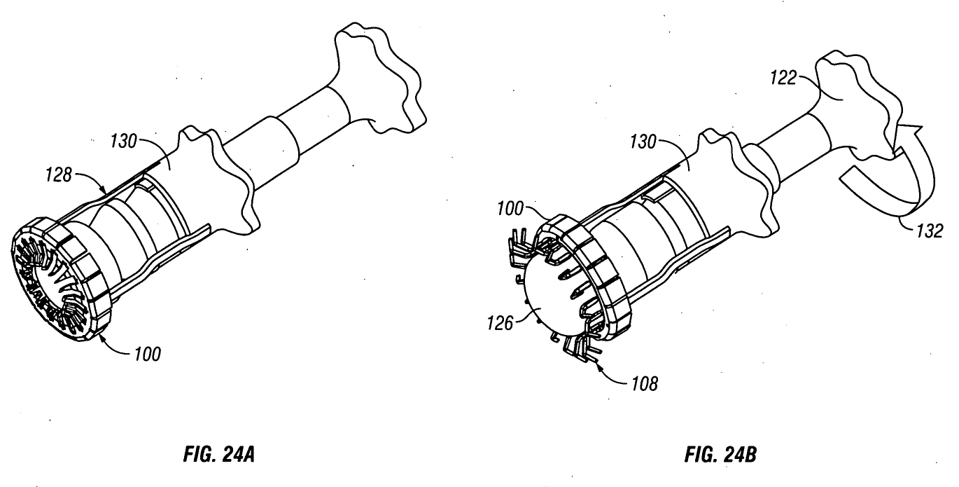

FIG. 17 shows a slightly different embodiment of the ring. FIG. 17 shows a ring 116 with substantially longer tines 108 to increase the amount of penetration into the tissue. Tine 106 may be shaped to be upward and substantially parallel to the flanges 104. In this present embodiment, there are three flanges 104 that have openings 118 therein. These openings may be used as anchor or attachment points for boot strap arms 128 (see FIG. 23A).

FIG. 18 shows how the ring 100 may be coupled to a stentless valve prosthesis P. The ring 100 is pressed to engage against a lower rim of the valve prosthesis P.

FIGS. 19A and 19B show cross-sectional views of the ring 100 engaged against the valve prosthesis P. FIG. 19A shows that the tines 106 engage the prosthesis at a level below that of the leaflets L. FIG. 19B shows a magnified view of one portion of the cross-section and shows that the tines 106 may be crimped to engage the valve prosthesis P. In one embodiment, the tines 106 engage the sewing annulus of the prosthesis P. The flange 108 may also be crimped radially inwards to better engage and grip the prosthesis P. In some embodiments, the tines 106 or some inner portion is not pushed radially outward, but instead only the outer flange 104 is pushed radially inward to engage and/or grip the valve prosthesis P. It should be understood that in other embodiments, the valve prosthesis may be molded to the ring 100.

Referring now to FIG. 20, one embodiment of a delivery device 120 for use in deploying the ring 100 may be used. A handle 122 is provided to deploy the fasteners or tines 108 into a target tissue. Some embodiments may be use a pistol-grip type handle to deploy the fasteners.

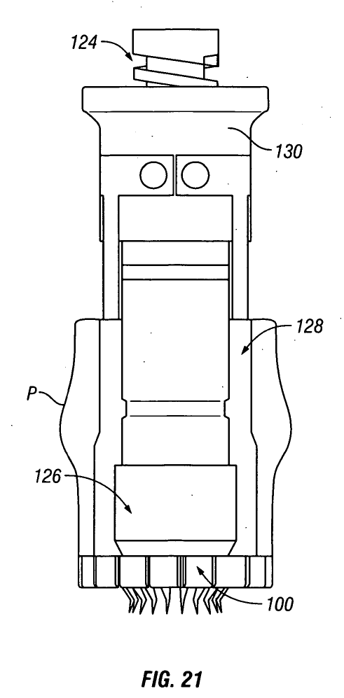

FIG. 21 shows a close-up view of one portion of the delivery device 120. The handle 122 may have a threaded portion 124 that will advance the shaft of the handle into the device when the handle is rotated in one direction. This will advance a plunger member 126 to engage the fasteners 108 and drive them into tissue. Supports or boot strap arms 128 may be used to hold the ring 100 until it is properly deployed and ready to be disconnected from the delivery device 120.

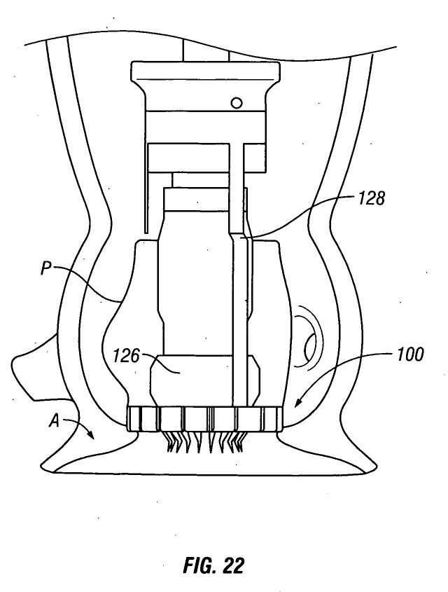

FIG. 22 shows pre-deployment of a ring 100 at a level of the native aortic annulus A. As seen in FIG. 22, the fasteners 108 have not been moved yet penetrate the tissue at the annulus A.

FIG. 23A shows one embodiment where the three boot strap arms 128 are positioned to attach to the interior of the ring 100. In this embodiment, the arms 128 and plunger member 126 are partially contained within the prosthesis. They may pass through the prosthetic leaflets.

FIG. 23B shows another embodiment where only the plunger member 126 passes through prosthetic leaflets and boot strap arms 128 attach on the outside of ring 100. The boot strap arms 128 may serve to maintain a fixed distance between the ring 100 and a housing portion 130. The housing portion 130 may engage the threaded shaft portion 124 and allow the plunger member 126 to drive through the ring 100 and advance penetrating members into the target tissue.

FIGS. 24A and 24B shows how the plunger member 126 may be advanced while the strap arms 128 holds the distance between the ring 100 and the housing 130. FIG. 24A shows the ring 100 with pre-bent staples or fastener 108 (prosthetic valve not shown for ease of illustration). FIG. 24B shows the three boot strap arms 128 that provide opposing force when knob or handle 122 turns to drive the plunger member which then drives staples or fasteners 108 into the annulus. The fasteners or staples are driven outward by the movement of the plunger member 126. FIG. 24B shows that in this embodiment, the rotation of the handle 122 as indicated by arrow 132.

FIG. 25 shows that some embodiments of the present invention may include element to minimize leakage between ring 100 and tissue such as the valve annulus. In the present embodiment, a Dacron felt gasket 134 may be provided for improved hemostatis (valve is not shown for ease of illustration). The gasket 134 may be positioned between the ring 100 and the prosthesis or in other embodiments, between the ring 100 and the annulus. The flanges 104 in this embodiment are smaller and are aligned with the tines 106. Some embodiments may have the flanges 104 and tines 106 offset to provide different gripping characteristics.

FIG. 26 shows the a Dacron felt gasket 134 with the prosthetic valve P in place.

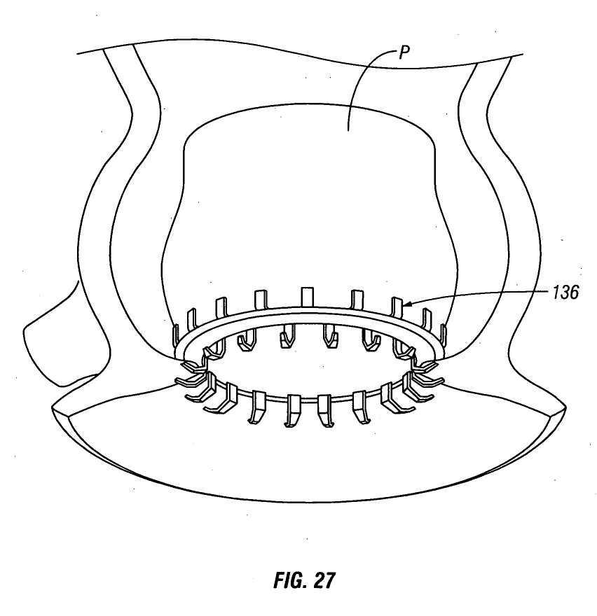

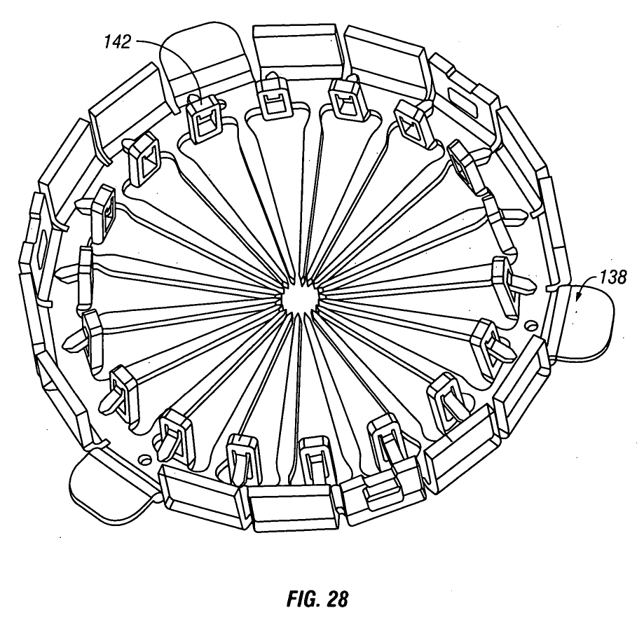

FIG. 27 shows a perspective view with a prosthetic valve P in place and a biodegradable ring portion 136. The biodegradable ring portion 136 may allow for initial rigidity and structural integrity during deployment. Over time, the ring portion 136 will dissolve away and allow the valve prosthesis P to conform better to the shape of the natural annulus.

FIG. 28 shows another embodiment of the present invention with locking cleats and tabs 138 that prevent the ring from traversing past the native aortic annulus, preventing deployment of the ring at an undesirable level. The tabs 138 may have different shapes such as but not limited to circular, oval, square, triangular, or any single or multiple combination of the above.

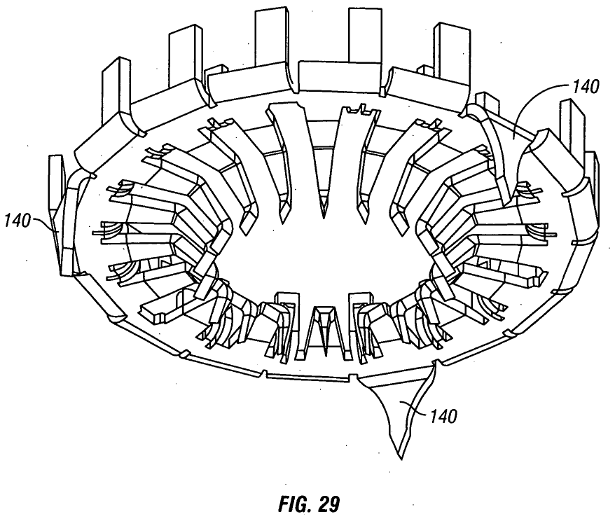

FIG. 29 shows yet another embodiment where the cleats 140 may be used to secure attachment of the ring to the annulus. The cleats 140 may be used to prevent the ring from traversing past the native aortic annulus, preventing deployment of the ring at an undesirable level.

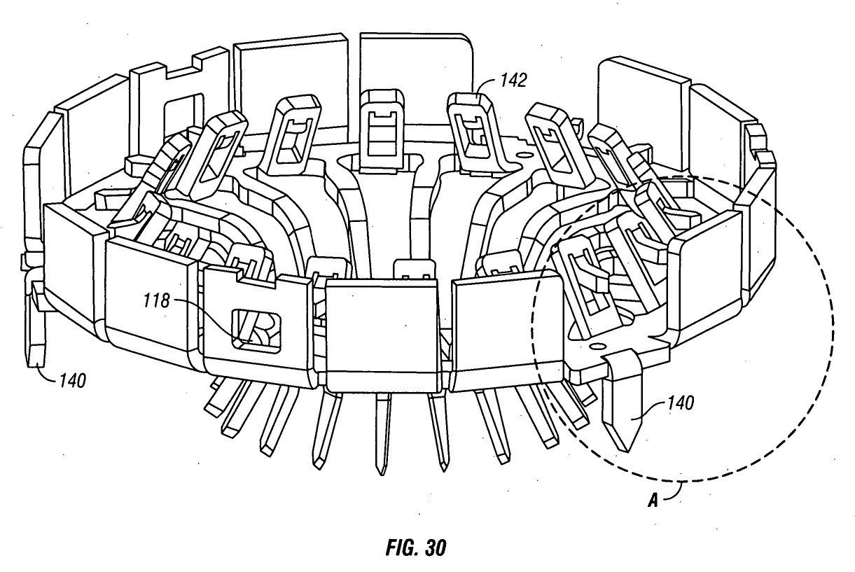

FIG. 30 shows yet another embodiment of the present invention wherein the ring 100 includes cleats 140 and valve attachment tabs 142 coupled to it. The tabs 142 include a inner piercing portion 144 that may be used to pierce into the prosthetic valve to better secure the valve in place. The portion 144 may act as a barb to pierce and hold the material in place. Optionally, the portion 144 may be integrally formed from a portion of the tab 142. The portion 144 may be angled downward towards the plane of the ring 100 to provide greater retention force on the valve prosthesis P.

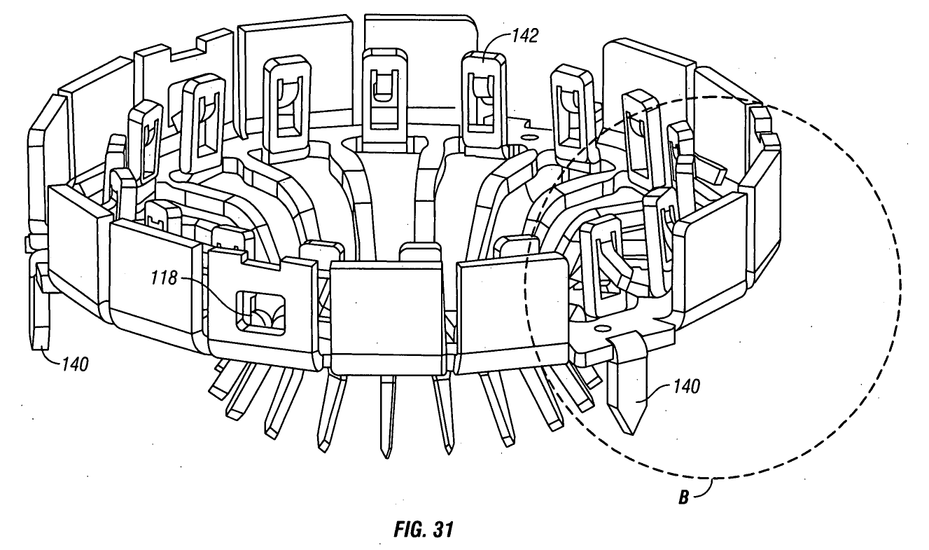

FIG. 31 shows the ring 100 of FIG. 30 with the tabs 142 moved to an upright, vertical position where the portions 144 will pierce, grip, and/or engage the valve prosthesis (not shown) and hold the prosthesis together with the ring 100.

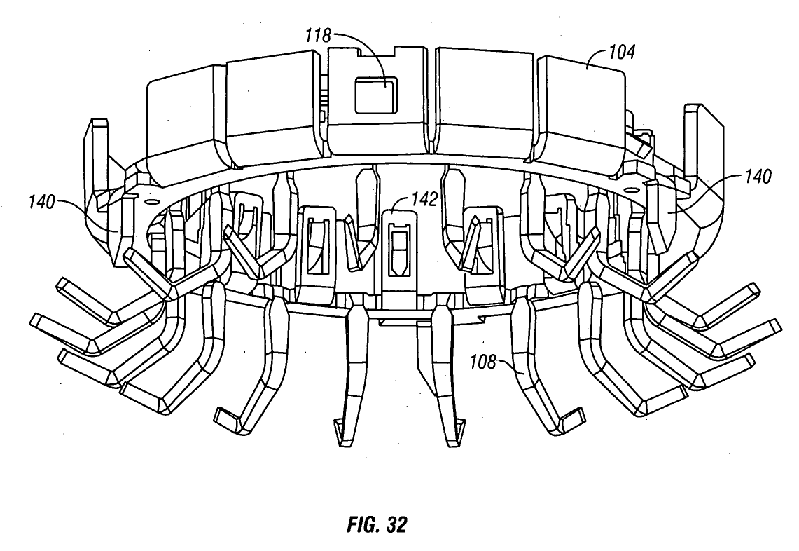

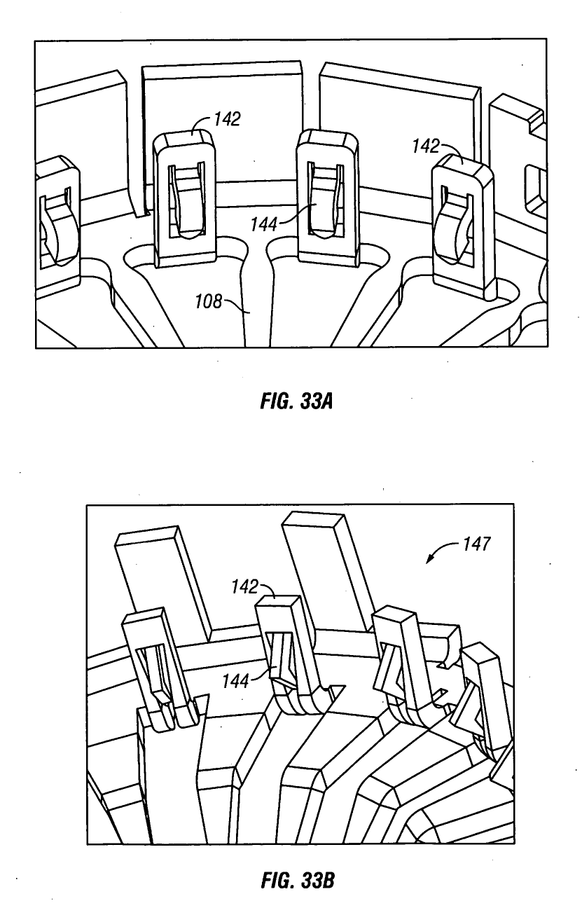

FIG. 32 shows the ring 100 with locating cleats 140 and staples 108 deployed. The tabs 142 with portions 144 are also positioned to grip the valve prosthesis P (not shown for ease of illustration).

FIG. 33A is a close-up perspective view another embodiment of the tab 142 with the attachment portion 144. Attachment portion 144 on the on staple ring 100 are used to fasten valve prosthesis to the ring. In the present embodiment, the attachment portion 144 is positioned inside the inner circumference defined by the plurality of tabs 142. The attachment portions 144 will be pushed through the opening 145 and engage the prosthetic valve P. FIG. 33B also shows a close of up another embodiment of a ring 147 with tabs 142 and attachment portions 144. Attachment points on the staple ring facilitate automatic attachment of valve prosthesis to staple ring using loading device (see FIG. 39).

Referring now to FIGS. 34A and 34B, another embodiment of the present invention will now be described. FIG. 34A shows a contoured ring 150 with a contoured shape to follow the shape of the native valve annulus. In one embodiment, the ring 150 has three rises to match the annulus. FIG. 34A is an embodiment where the staples 152 are located in the non-rise portions of the ring 150. The cleats 154 are used to locate the annulus and allow for the ring 150 to seat securely in place. The ring 150 has a channel portion 156 that is configured to receive a vertical portion of a valve prosthesis P. The channel portion 156 may be crimped onto at least a portion of the valve prosthesis P. Optionally, the channel may be defined by a plurality of tabs 104 that may be used to define the channel. In other embodiments, the valve prosthesis may adhered, molded, or integrally formed with the ring 150. Any of the embodiments of the rings described in this application may be adapted to have a contoured configuration as shown in these figures.

FIG. 34B shows a perspective view from the underside of the ring 150. The staples 152 have been bent to swing outward, away from the inner circumference towards the outer circumference of the ring 150 to engage tissue. FIG. 34B also more clearly shows the cleats 154 used to engage the native annulus and seat the ring 150. FIG. 34B also more clearly shows the three rises 158 that are part of the contour of the ring 150. As a nonlimiting example, each rise has a set of cleats 154. Other embodiments may have cleats 154 only at two rises or one rise. Still other embodiments may vary the number of cleats 154 at each rise. By way of nonlimiting example, some may have four, three, two, or one. Of course, the ring 150 does necessarily have three rises and may be designed to follow the contour found at the target site. This will provide improved seating of the valve and less strain on the native annulus.

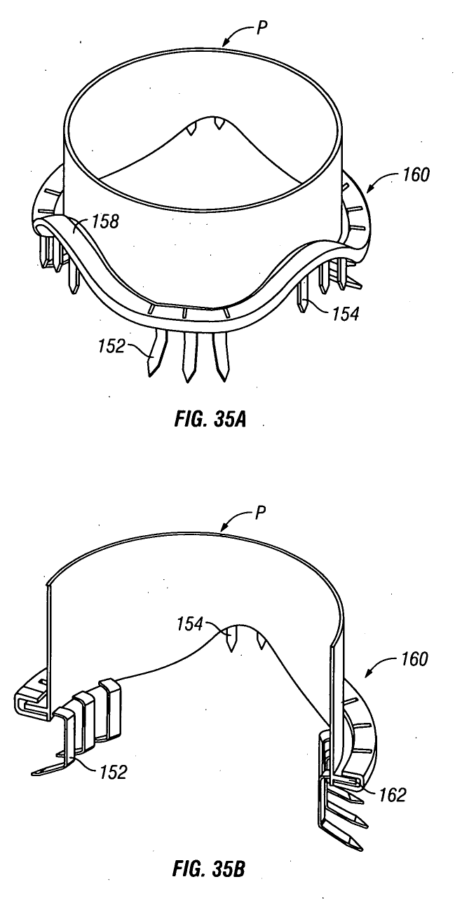

Referring now to FIGS. 35A and 35B, yet another embodiment of a contoured ring will now be described. The contoured ring 160 of FIG. 35A is configured for use with a stented contoured valve prosthesis. Specifically, FIG. 35A shows a contoured ring 160 with a channel used with a prosthesis P with a contoured ring which may be similar to a sewing ring. This enables more traditional stented valves to be used the a ring 160. In some embodiments, the valve prosthesis may be folded on and/or crimped on the ring 160. The prosthesis may come pre-loaded onto the ring 160 or other embodiments may have the surgeon load the valve prosthesis onto the ring 160.

FIG. 35B is a cross-sectional view showing a channel 162 for receiving the valve prosthesis P. In some embodiments, the channel 162 may be crimped to more tightly grip the prosthesis P.

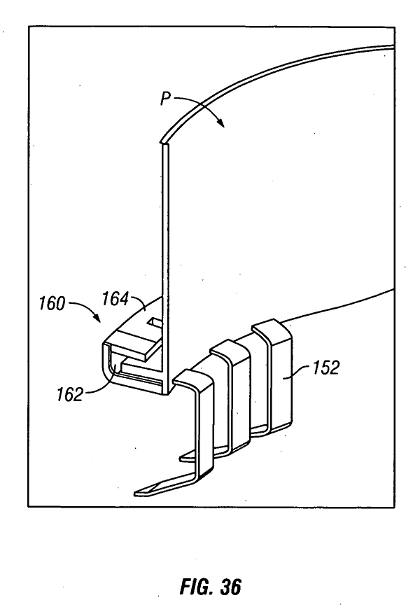

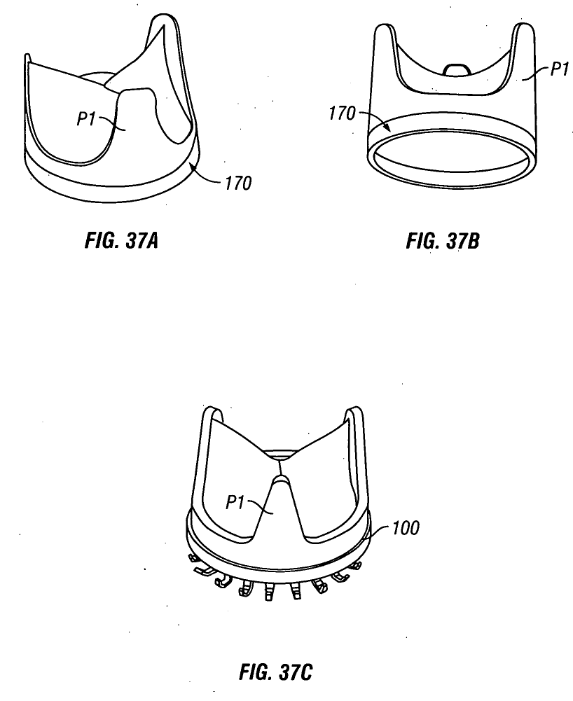

FIG. 36 more clearly illustrates how the channel 162 may be used to secure a stented portion of the valve prosthesis P in place. As seen in FIG. 36, tabs 164 may also be used to secure the ring in place.

FIGS. 37A and 37B shows a stented prosthesis modification to accommodate a staple ring attachment. As seen in FIG. 37A, the bottom annular portion of the stented prosthesis P1 may have an ring portion designed to be received by the ring 100. This a new valve prosthesis designed for use with the ring. Some embodiments may view this as an extension 170 which may be integrally formed or adhered to the valve prosthesis P1. FIG. 37C shows a ring 100 receiving therein the valve prosthesis P1. The extension may also be contoured to fit with a ring. The sewing ring on a conventional valve prosthesis is removed on an existing valve and the extension is used to facilitate coupling.

FIGS. 38A-38C show a stentless prosthesis modification to accommodate a contoured staple ring. In FIG. 38A, the stentless valve prosthesis P2 is shown with an extension that has a contoured lip 172 to follow the contour of the ring 150. FIG. 38B is a side view of the prosthesis P2. FIG. 38C shows a bottom up view of the prosthesis P2. The leaflets 174 are visible from this angle.

Referring now to FIG. 39, devices are shown for loading a stentless valve to the staple ring. A valve prosthesis loading device 180 is shown. Loading device 180 includes a plurality of slots 182 for securing sutures. A staple ring loading dock 184 is provided on one end of the loading device 180. Some embodiments may further include slots 186 are for a deployment device (not shown) to grip the ring via openings 118 on the ring. The center area of the loading device 180 is where the ring will sit.

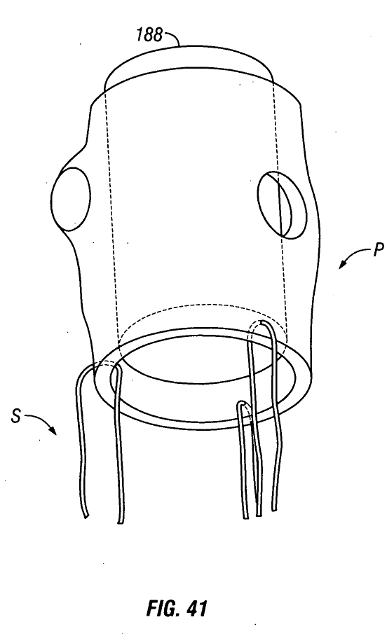

FIG. 40 shows a view from the underside of the valve prosthesis loading device 180.

Referring now to FIG. 41, leaflet protector 188 may be used when inserting deployment device (green). Leaflet Protector 188 allows for safe insertion of deployment device in Step 4. In one embodiment, the leaflet protector 188 is deployed up from the underside of the valve prosthesis. As mentioned, this protects the leaflets. The anvil for the staples is then lowered through the inside of the protector 188. Once the ring is engaged, leaflets are pulled out into the open space inside the delivery device. Sutures S are shown coupled to the valve prosthesis. They are first positioned on pre-located positions on the graft.

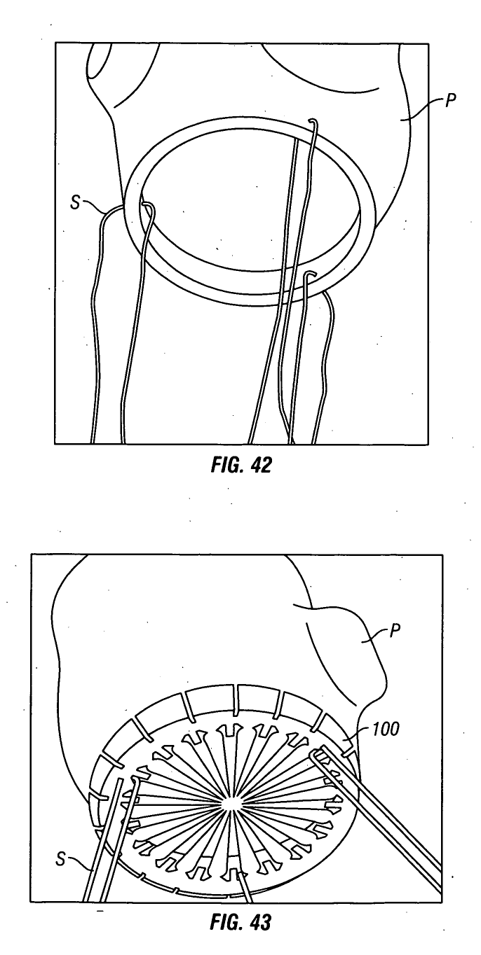

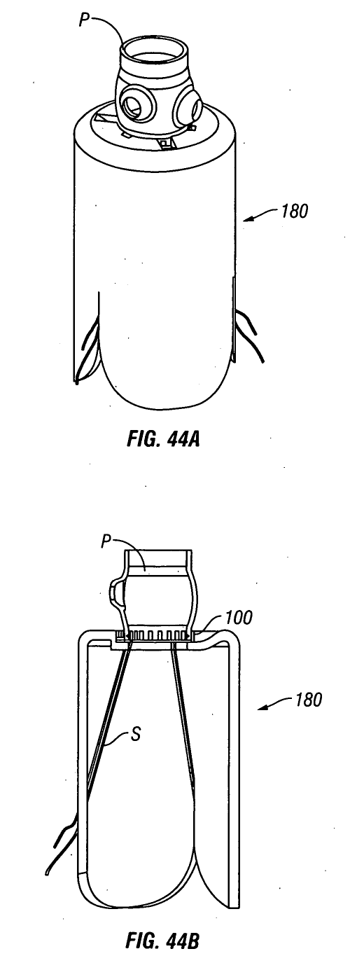

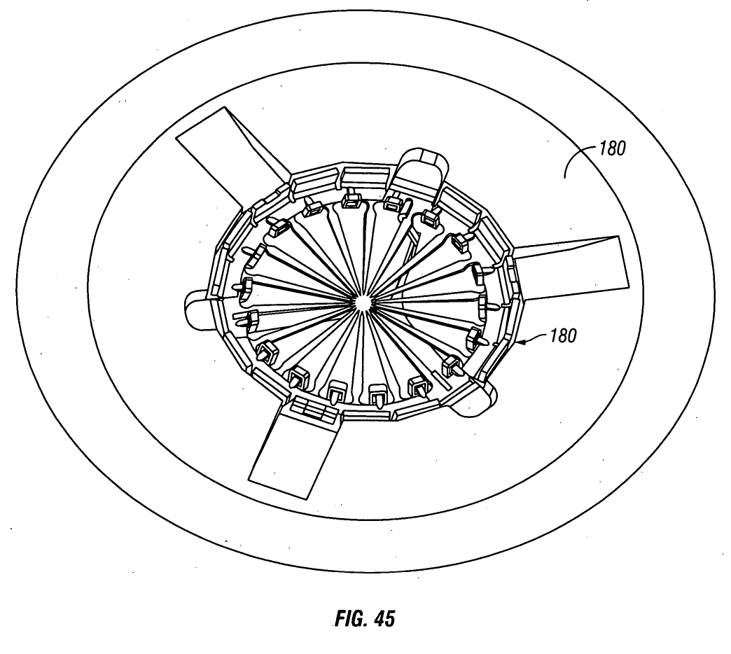

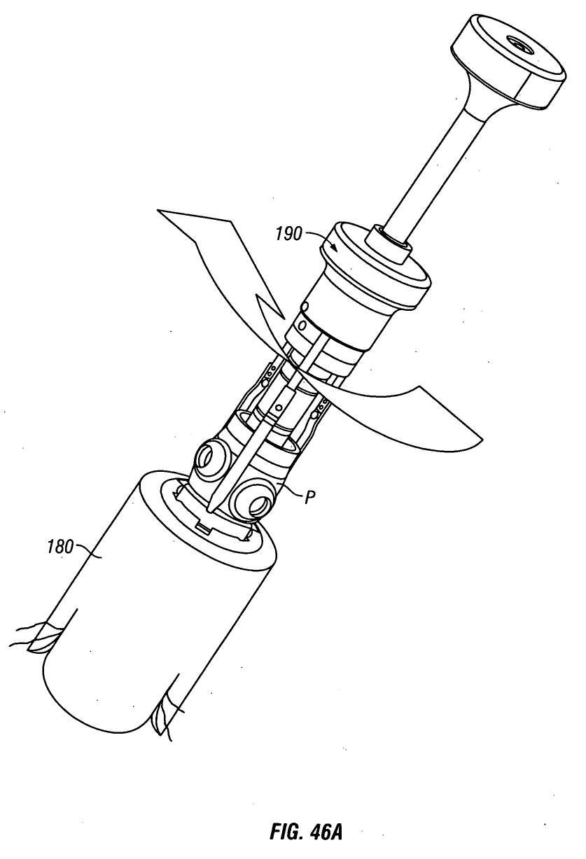

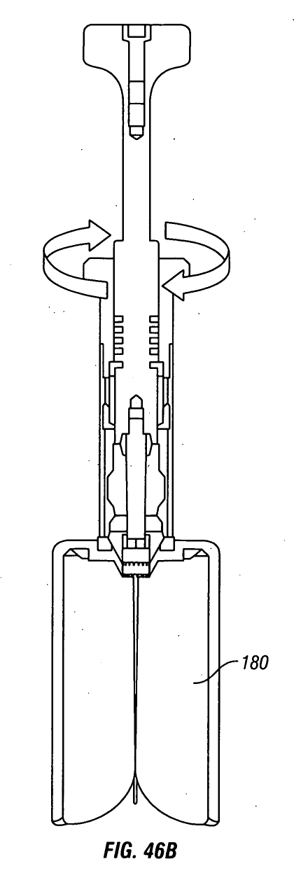

Referring now to FIGS. 42-46, the method for using the loading device 180 to load a valve prosthesis on a deployment device may include the following steps. 1) Sutures S are inserted onto prosthetic valve P (see FIG. 42). 2) Sutures pass S through Staple Ring 100 (see FIG. 43). 3) Sutures are fastened to loader slots (FIG. 44A). 4) Deployment Device may be inserted through prosthetic valve orifice to fasten Ring to the deployment device (see FIGS. 46A and 46B). In one embodiment, clockwise turning of second knob 190 deploys attachment points at the inter flanges onto sewing ring of prosthesis. This pushes the attachment points 144 into the valve prosthesis. FIG. 45 shows the ring 100 mounted on the loading device ready to be engaged with the deployment device (the valve prosthesis is not shown in the embodiment).

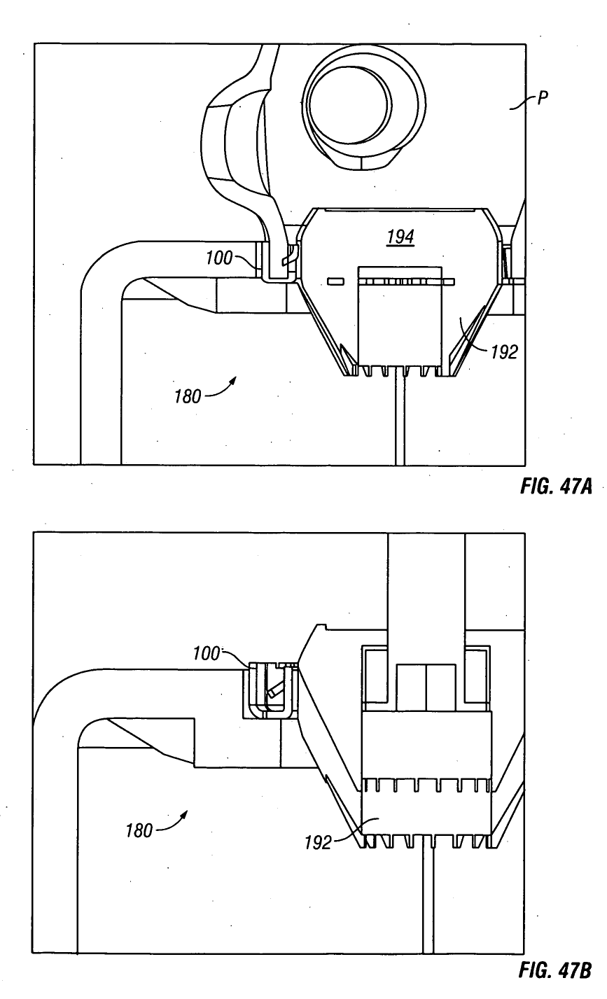

FIG. 47A shows how the ball anvil 192 can be lowered as indicated by arrow 194 to drive the attachment points 144 into the valve prosthesis P. FIG. 47B shows the ball anvil 192 in a first position and a second position where the anvil 192 has been lowered and presses the attachment points into the valve prosthesis.

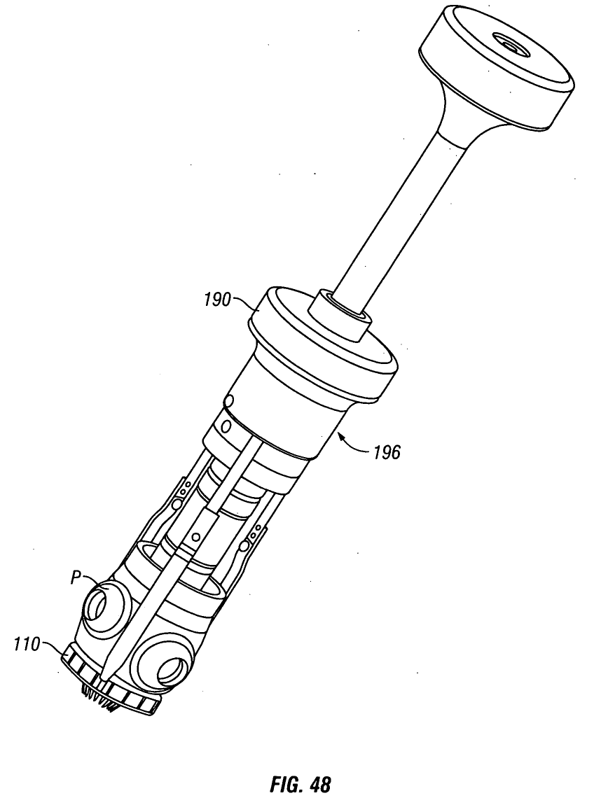

FIG. 48 shows a deployment device 196 with the valve prosthesis P and ring 100 attached.

FIG. 49 shows an off pump adaptation for a staple ring deployment device 200. The deployment device 200 has an extended shaft portion 202. The off pump device access the valve via the aorta. The length of the shaft portion 202 is sized so that the staple ring may be delivered minimally invasively to the target site.

FIG. 50 shows a cross-sectional view of the off pump device 200. The device 200 is substantially similar to the other deployment devices herein. Some embodiments are sized so that the knob 204 and handle 206 are outside the patient's body during staple ring delivery and deployment.

FIG. 51 shows a segmented staple ring 210. This embodiment of the invention is directed at using a non-rigid, segmented ring to attach a prosthetic valve to the tissue. The segmented ring 210 allows for greater flexibility for seating purposes. In FIG. 51, the individual segments 212 are designed to have two staples 108. Other designs may vary the number of staples 108 per segment.

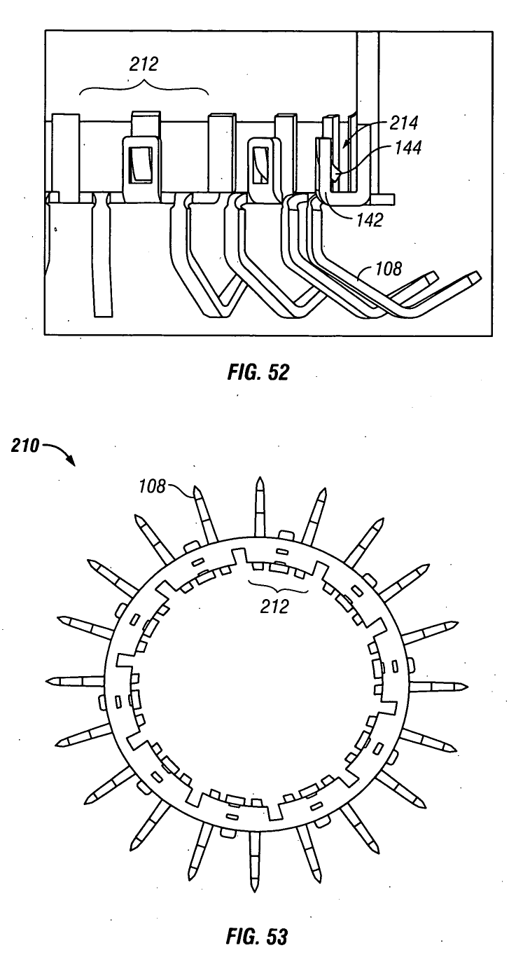

FIG. 52 shows the embodiment showing the segmented ring coupled to prosthetic valve annulus that fasten in this groove 214 similar to other staple ring.

FIG. 53 shows a top view of the ring 210 having a plurality of segments 212 attached together to form a ring. The segments 212 may be attached an annular device that maybe biodegradable. In other embodiments, the annular device 216 is made of a pliable material. In other embodiments, the annular device 216 is made of a biodegradable or dissolvable material such as but not limited to polyglycolic acid.

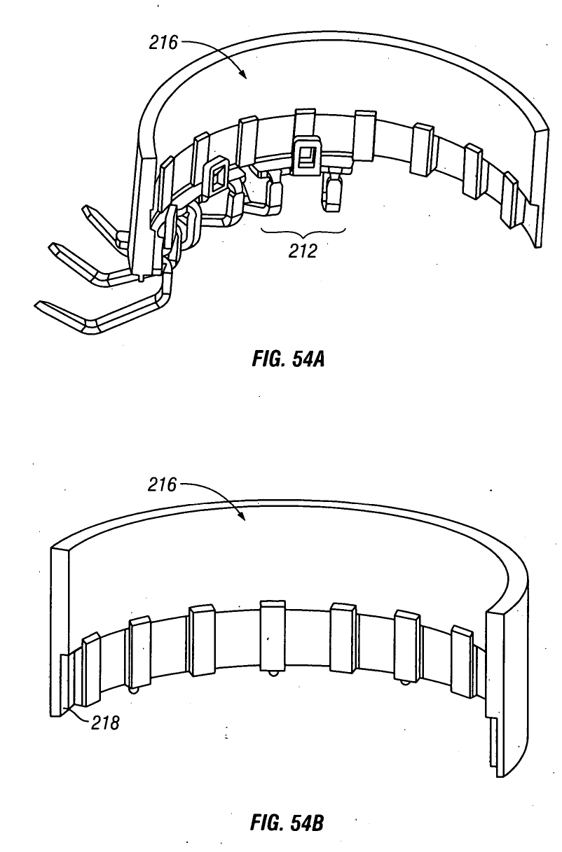

FIG. 54A shows how the segments 212 can be loaded into the annular device 216. FIG. 54B shows that slots 218 are formed in the annular device 216 for receiving the segments 212.

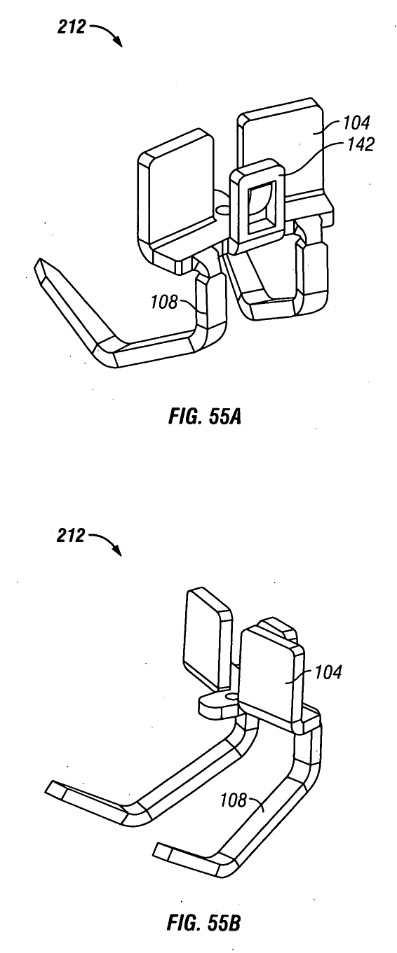

FIGS. 55A and 55B show enlarged views of one embodiment of a segment 212 for use with the annular device 216. Each segment 212 is designed to engage the annular device 216 and to engage a valve prosthesis P that will be coupled to the segment 212.

FIG. 56 shows one embodiment of an imaging device 220 for off pump aortic valve replacement using a staple ring device. The intra-staple ring device for 3D imaging of the aortic root will better facilitate the placement of the valve prosthesis P. The imaging device 220 has an imaging element 222 that is mounted near the distal end of the elongate device.

FIG. 57 shows a close-up view of the imaging element 222 sized to pass through the deployment device 200.

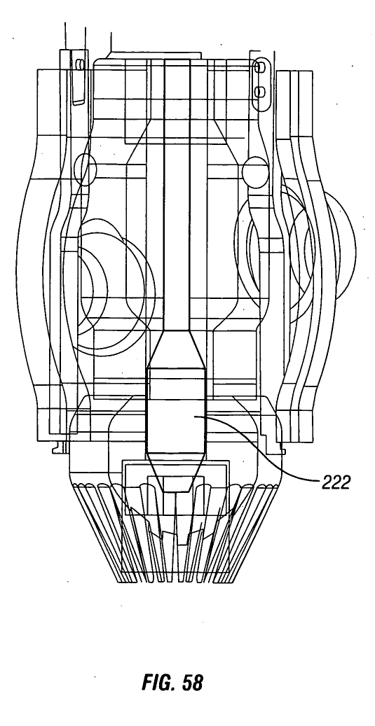

FIG. 58 shows how the imaging element 222 is sized with a diameter to fit within a central lumen of the deployment device 200. This allows the imaging element 222 to be positioned at or near the native valve annulus to image the position of the tissue relative to the delivery device and/or the attachment ring 100.

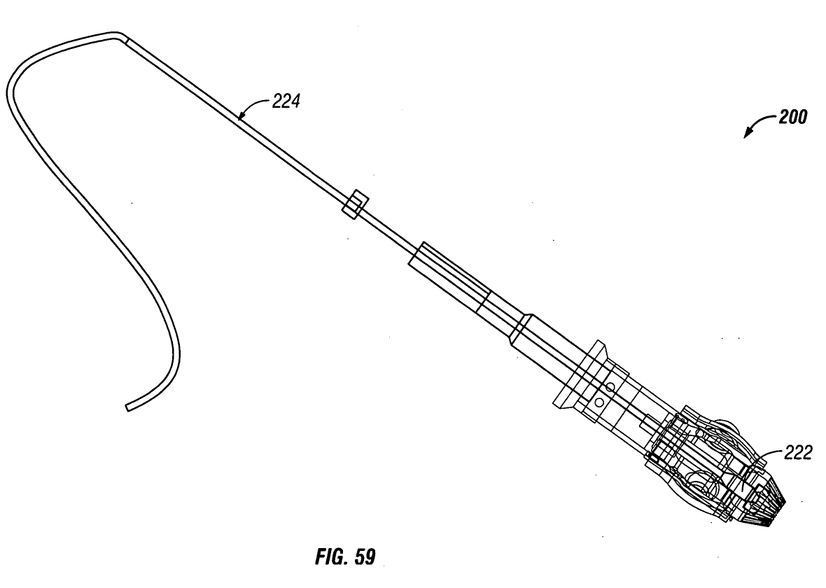

FIG. 59 shows how the proximal portion of the imaging device 220 may extend outside the deployment device 200 in a manner sufficient to allow for rotation and translation of the device 220 to obtain the proper position for imaging. The rotation and/or translation of the imaging device 220 may allow for construction of a 3D image to more accurately locate the valve prosthesis. Using a imaging device 220 at the level of the valve prosthesis allows for a much faster image capture. In one embodiment, it takes less than 60 seconds to obtain data to generate a 3D image. In some embodiments, the imaging element 222 may extend out the distal end of the deployment device 200. This allows imaging of tissue prior to engagement by the deployment device 200. In one embodiment, a proximal shaft portion of the device 220 may have marks, indentations, or protrusions 224 so that a user will know how far the element 222 may be extending outside the device 220 or provide position information generally.

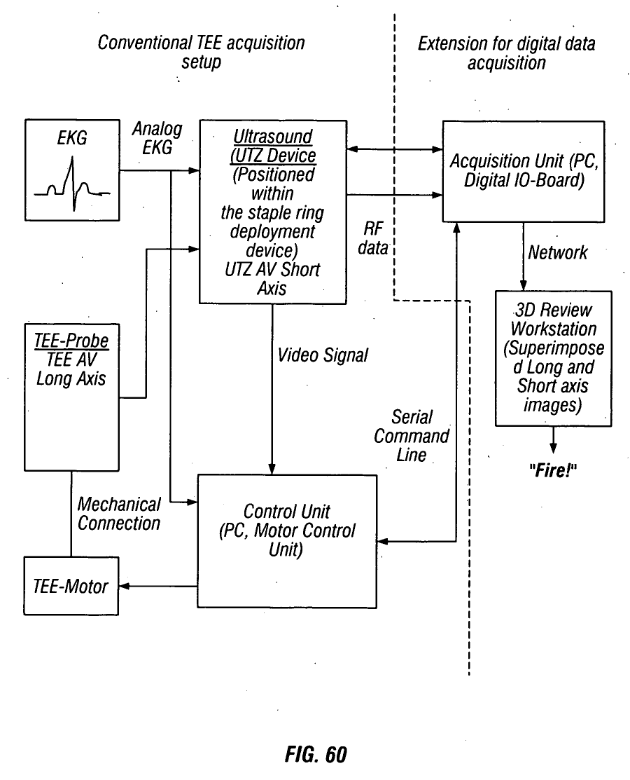

FIG. 60 shows a flow chart of a method according to the present invention. The improvement of the present invention involves the use of probes to create a 3D image. The present method may use an ultrasound (UTZ) Device (Positioned within the staple ring deployment device) UTZ AV Short Axis. The device may use a TEE-Probe for TEE AV Long Axis. Both long and short axis image are assembled to provide a 3D image. A 3D review workstation is used to superimposed Long and Short axis images. After the image is obtained, the device is fired to deploy the staple ring at the target site.

FIGS. 61A and 61B show how a delivery device 200 according to the present invention may be used staple ring modifications for mechanical bileaflet valve 230. As will be described in further detail below, the mechanical valve 230 is configured to have a plurality of fasteners or staples 108 to secure the valve to the native annulus. FIG. 61B shows the mechanical valve 230 with the fasteners 108 in a deployed position. The mechanical valve 230 may be coupled via boot strap arms 128.

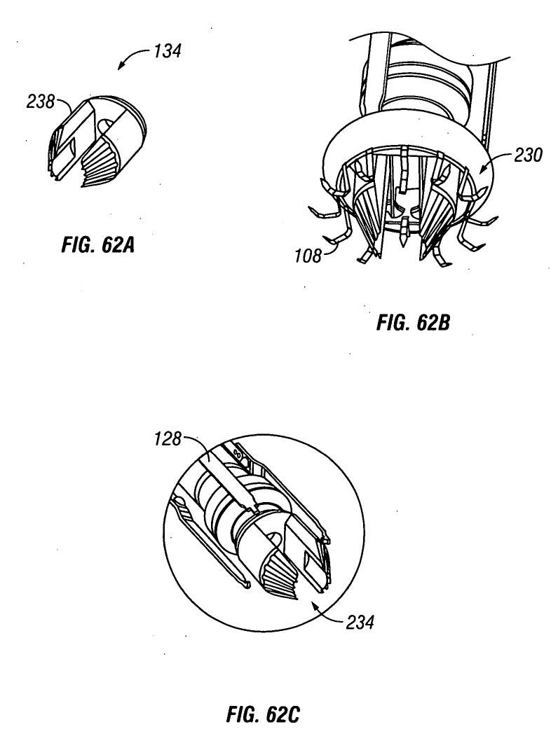

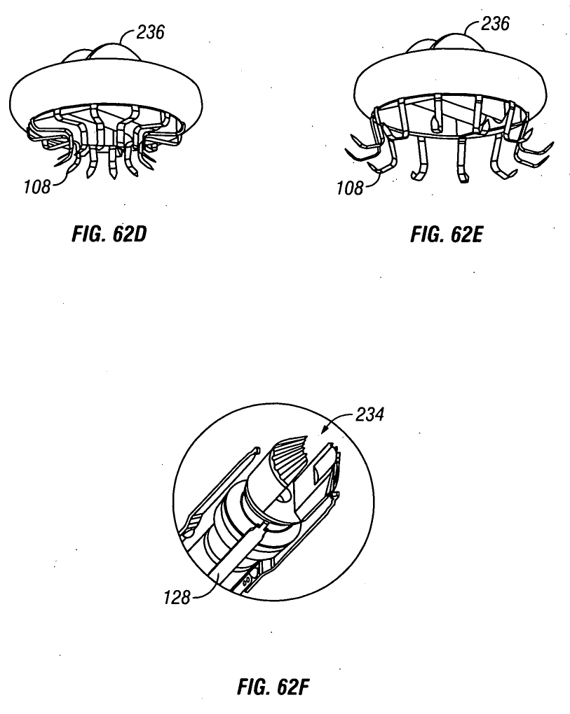

FIG. 62A is close-up view of one embodiment of an anvil 234 for use with a mechanical valve. The plunger or anvil 234 is a solid piece that is grooved inside to accommodate the valve leaflets 236 of mechanical valve 230 (leaflets shown in FIG. 62D). The groove 238 allows the anvil 234 to extend down and engage the fasteners 108 without damaging the valve leaflets which are folded upwards. FIG. 62C shows the fasteners 108 in pre-deployed configuration and FIG. 62D shows the fasteners 108 in a fully deployed configuration.

As seen in FIG. 62B, when the plunger or anvil 234 is fully extended, only about half of the fasteners 108 are in contact with the plunger. Thus, to deploy all of the fasteners 108, the plunger 234 may be retracted, rotated 90 degrees (FIG. 62F) and then lowered back down to engage the remaining fasteners 108 and push them outward.

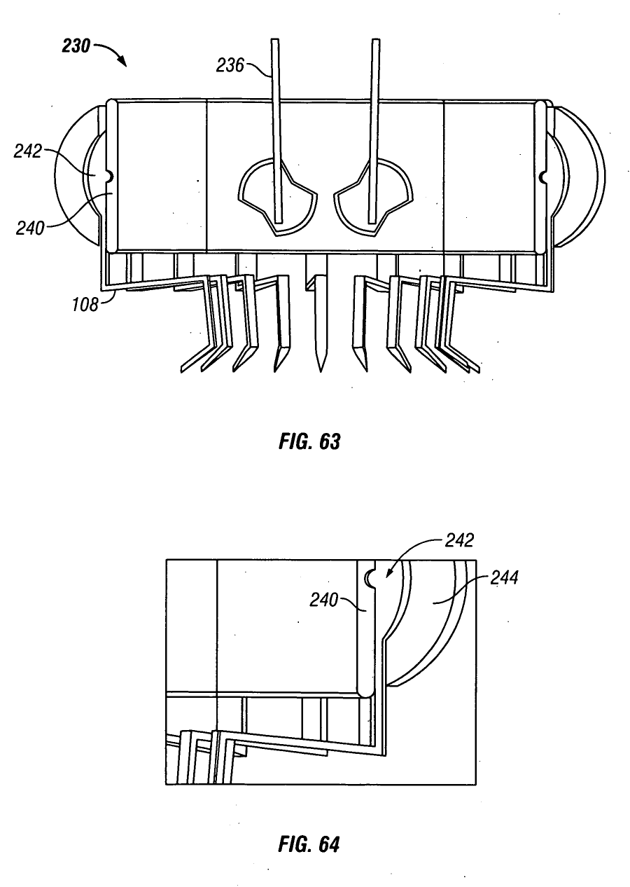

Referring now to FIG. 63, staple ring has been integrated within the rotating housing of mechanical valve prosthesis. The inner housing 240 is rotatable relative to the ring 242 with the fasteners 108. A sewing ring 244 or other member may form the outer housing which helps seal against the native valve tissue. The rounded portion is the current sewing ring provides a fabric seal with the tissue. The elements are also shown in greater detail in FIG. 64.

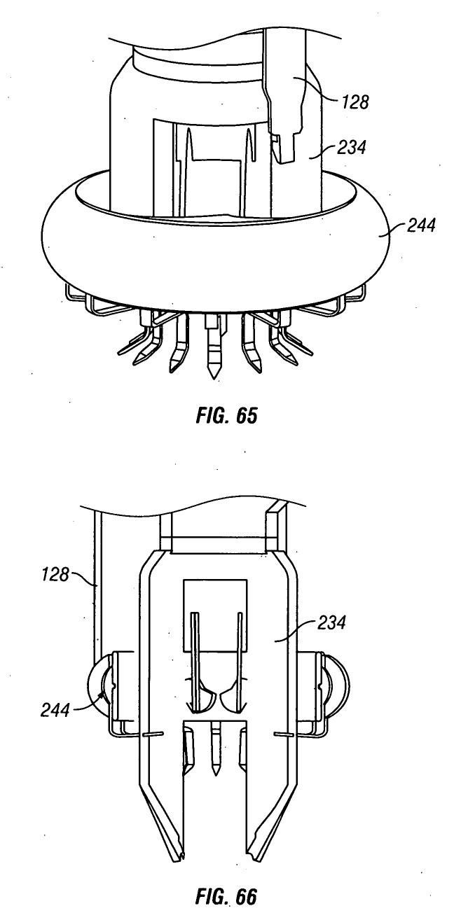

FIG. 65 shows the plunger 234 passing through the mechanical valve 230.

Referring now to FIG. 66, after the initial pass to deploy about half the fasteners, the plunger is drawn back. The inner portion of the valve with the leaflets rotates relative to an outer housing 244 and ring 242. The plunger is then pushed back down to deploy the other half of the fasteners 108.

Referring now to FIG. 67, yet another embodiment of a ring 300 is shown with a plurality of fasteners 302 and another set of fasteners 304. In one embodiment, the ring 300 is made from one piece of material such as but not limited to titanium, steel, or stainless steel. The appropriate cuts are made to form the staples or fasteners 302 and 304 that attach the valve prosthesis and the staples that attach the valve prosthesis to the annulus respectively. The single linear piece is then rolled and welded into the circular configuration. The staple members are then bent to the appropriate pre-deployed angles. As seen in FIG. 67, some fasteners 302 are angled upward and other fasteners 304 are angled downward. As seen, in this particular embodiment, the fasteners 302 and 304 may each have two or more bends. The fasteners 302 and 304 all attach to the same edge of the ring 300. In the present embodiment, the fasteners 302 and 304 attach to a bottom edge of ring 300.

As seen in FIG. 68, the ring 300 also include tabs 310 for resting against a target tissue to hold the ring 300 in proper position. The ring 300 may have one, two, three, four, or more tabs 310. They may be rectangular, square, round, triangular, hexagonal, polygonal, or other shaped.

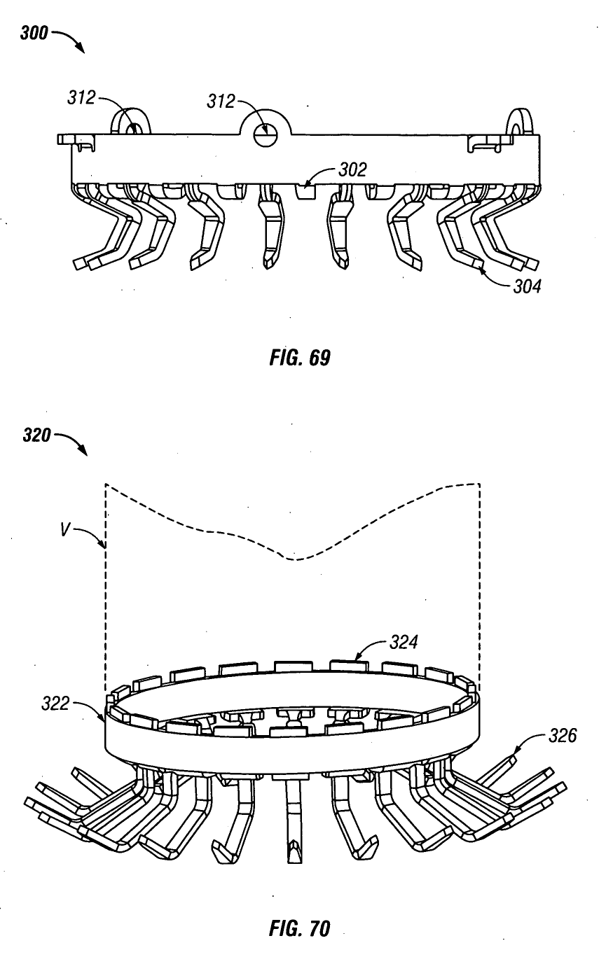

As seen in FIG. 69, the ring 300 also includes a plurality of attachment points 312 that allow holders on the delivery device to engage the ring 300. The ring 300 may have one, two, three, four, or more attachment points 312. The openings in the attachments points 312 may be rectangular, square, round, triangular, hexagonal, polygonal, or other shaped.

Referring now to FIG. 70, yet another embodiment of the present invention will now be described. This ring 320 includes a flexible, annular portion 322 that holds a plurality of individual fastener segments 324 together. The annular portion 322 may be a flexible ring holder made from elastomer, thin metal, silicone, or plastic. The segments 324 may include tines 326 which will pierce into the target tissue to secure the ring 320 in place. It should be understood that each segment 324 may be similar to the segments shown in FIGS. 55A and 55B where the may be more than one tine 326 per segment. Each segment may also include a portion 142 for securing the prosthetic valve to the ring. In some embodiments, the prosthetic valve V may be molded directly to the ring 320 as shown phantom.

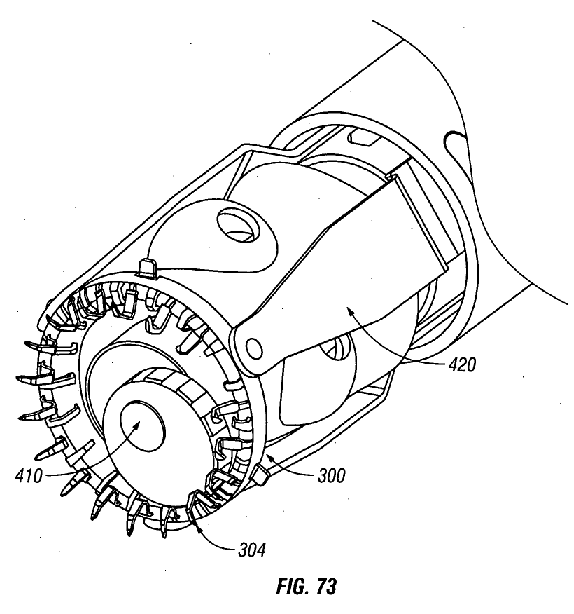

Referring now to FIG. 71, another embodiment of a delivery device for a ring 300 attached to a valve prosthesis. FIG. 71 shows the handle of delivery device 400. A knob 402 is rotatable to move one set of fasteners 304 on the ring 300 into the target tissue. As will be see in FIG. 73, the knob 402 is coupled to a shaft that is offset from the longitudinal centerline of the device. This offset configuration allow the distal end of shaft to engage the fasteners and exert radially outward (and optionally, radially outward and upward) force to move fasteners 304 into place. The knob 402 may include a marker. to show the end that is engaging fasteners. In this deployment device, the fasteners are not deployed by passing the anvil thru the annulus. The knob 402 at the end of the blue embodiment has a rod that is displaced from the center of the entire assembly. When the orange knob at the top of the assembly is turned it displaces the orange anvil off center and drives the staples into the annulus. This is repeated a few times to deploy all staple in a similar fashion. This would take about 10 seconds.

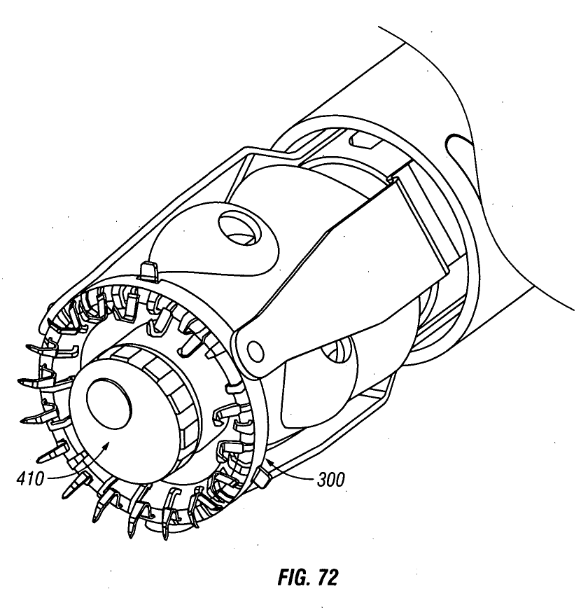

FIG. 72 show a distal end of the device of FIG. 71. The disc 410 on coupled to that shaft coupled to knob 402 is shown.

FIG. 73 shows that when the knob 402 is rotated, the disc 410 is now offset from its center position and now engages the fasteners 304. The center shaft 412 is then rotated by turning knob 414 seen in FIG. 71. This rotates the disc 410 around the entire circumference of the ring 300, engaging fasteners 304 as the shaft 412 is rotated. This provides radially outward force to move the fasteners to engage target tissue. As seen in FIG. 73, holders 420 engage that attachment points 312 on the ring 300. The holders 420 are spring mounted and can be pressed radially inward to engage the points 312. The disc 410 may have slots 411 to engage the fasteners 304. The edge of the disc 410 may also be beveled to be angled to provide a 45 degree or other angle so that outward and upward force maybe provided.

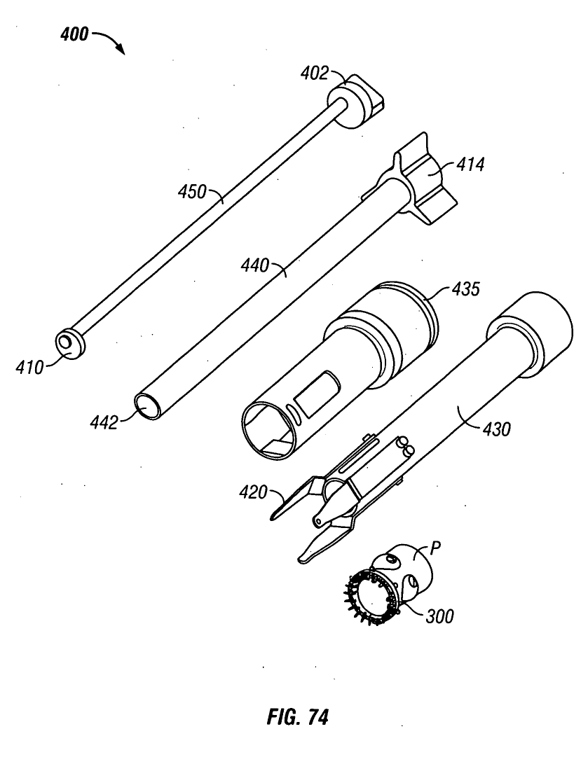

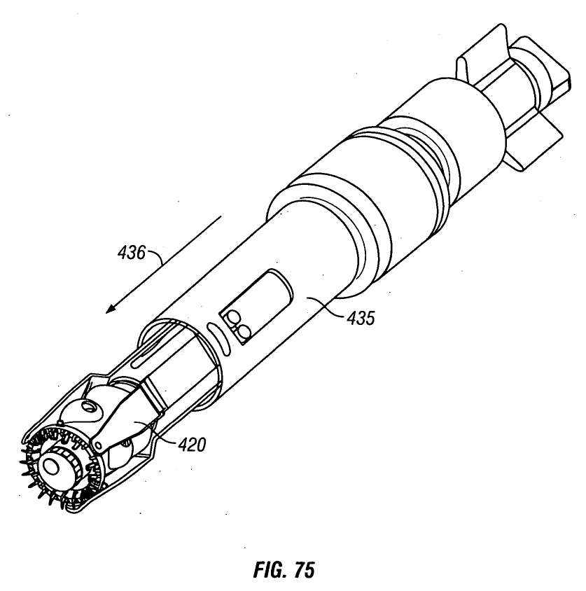

FIG. 74 shows an exploded view of the device. A valve prosthesis P with a ring 300 is provided. A shaft 430 with deflectable holders 420 is provided. Shaft 430 slides within housing 435 which will press holders 420 radially inward to engage the ring 300. shaft 440 is coupled to knob 414 which is turned to “walk” disc 410 around the ring 300. The shaft 440 includes a lumen 442 that is radially offset from the longitudinal centerline of the shaft. The shaft 450 is coupled to disc 410 and knob 402.

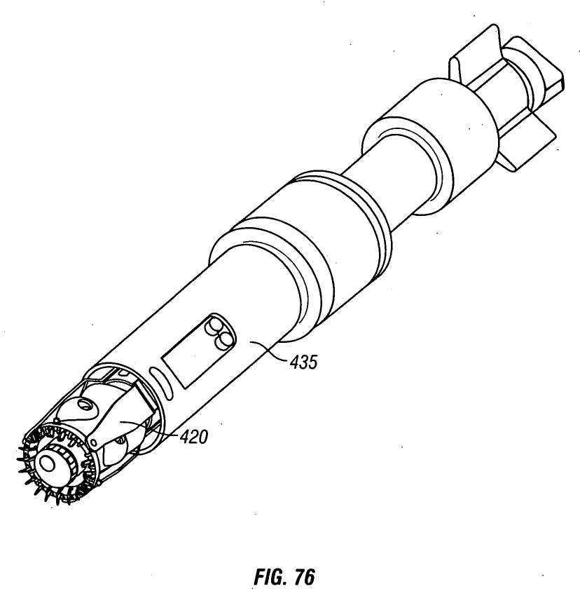

FIG. 75 shows how the housing 435 is slidable downward as indicated by arrow 436 so that the holders 420 then are pressed radially inward to engage the ring 300.

FIG. 76 shows the housing 435 slid in placed to move the holders to engage ring 300. The movement of housing 435 allows the ring 300 to be loaded onto the delivery device 400 and then released once the valve and ring 300 are secured at the target tissue.

Referring now to FIG. 77, a kit 500 according to the present invention will be shown. The kit 500 may comprise of a valve prosthesis delivery device 502, a valve prosthesis 504, and instructions for use (IFU) 506 setting forth the method of delivery or attachment. The kit may also include a container 510 sized to house the valve prosthesis delivery device, the valve prosthesis, and the instructions for use. In some embodiments, the prosthesis 504 may be separate from the kit and not mounted directly on the device. Other embodiments may have only the prosthesis 504 and not the delivery device 502.

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, with any of the above embodiments, a prosthetic valve or a graft may be premounted on to the apparatus. With any of the above embodiments, the apparatus may be configured to be delivered percutaneously or through open surgery. With any of the above embodiments, the horizontal cross-section of the shaped plunger member 40 may include portions that include but are not limited to being oval, polygonal, square, rectangular, or any combination of the above. With any of the above embodiments, sequential fastener deployment may be achieved by having grooves along the member 40 so that some fasteners are in the groove and not pushed out until the member 40 is advanced forward. The member 40 may also be keyed so that it only aligns with the ring 10 in a certain orientation. With any of the above embodiments, sizing of the valve prosthetic and shaped member 40 may be matched. This may involve changing the member 40 to match the valve prosthetic size. The number of fasteners 12 on the ring may also be varied. By way of example and not limitation, some embodiments may only have 3-5 fasteners. Others may have more than 10, 15, 20, 25, 30, 25, 40, 45, 50, ore more fasteners per ring. Any of the above embodiments may be adapted for use in attaching tissue-to-tissue such as but not limited to an end-to-side anastamosis procedure where the vessel to be attached may be coupled to the ring with a plurality of fasteners. The ring would then be coupled an opening on another blood vessel.

The publications discussed or cited herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. All publications mentioned herein are incorporated herein by reference to disclose and describe the structures and/or methods in connection with which the publications are cited.

Expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A device for use with a valve prosthesis, the device comprising:
a ring; and
a first set of fasteners attached to said ring, wherein said fasteners are deformable to move from a first position to a second, tissue engagement position;
wherein said ring defines a groove for receiving at least one portion of the valve prosthesis.

2. A delivery system for use with the device of claim 1 wherein the system further comprises a shaped plunger member, wherein the shaped plunger member is movable along a longitudinal axis of the device and has a diameter sufficient to move said fasteners from a first position to a second, tissue engagement position.

3. A delivery system for use with the device of claim 1 further comprising a support device is expandable from a compressed configuration to an expanded configuration, said support device used to position the ring.

4. A delivery system for use with the device of claim 1 further comprising a support device is expandable from a compressed configuration to an expanded configuration, said support device used to trap tissue between itself and the ring.

5. The delivery system claim 4 wherein the support device is formed from a plurality of elongate support elements extending radially outward from a central disc, said support elements movable from a first position to a second, expanded position.

6. The delivery system of claim 4 wherein the support device is configured to be engaged by a shaped plunger member have a circumference sized to move support elements on the support device from a first position to second, expanded position.

7. The delivery system of 6 wherein the shaped plunger member is sphere-shaped having a diameter sufficient to move said support element to the second position.

8. The delivery system of claim 6 wherein the shaped plunger member is mounted to shaft that is slidably mounted within a shaft coupled to the support device, said shape member movable relative to the support device.

9. The delivery system of claim 6 wherein the shaped plunger member is mounted to shaft that is slidably mounted over a shaft coupled to the support device, said shape member movable relative to the tissue connection device.

10. The device of claim 1 wherein the fasteners have a curved distal portion.

11. The device of claim 1 wherein the fasteners have a sharpened distal tip.

12. The device of claim 1 wherein the fasteners have a blunt distal tip.

13. The device of claim 1 wherein the fasteners are made of stainless steel.

14. The device of claim 1 wherein the fasteners in the first position extend in part towards the inner circumference of the ring.

15. The device of claim 1 wherein the fasteners have a first portion that is straight and a second portion that is curved.

16. The device of claim 1 wherein the fasteners have a first portion extending in a first direction and a second portion extends in a second direction.

17. The device of claim 1 further comprising a mesh or a polymer membrane coupled to said fasteners to minimize fluid leakage between the valve prosthesis and the target tissue once the valve prosthesis is delivered.

18. A method for attaching a valve prosthesis to a target tissue, the method comprising:
providing a ring having a portion defining a groove and a plurality of fasteners coupled to the ring;
placing a portion of the valve prosthesis in the groove on the ring;
securing the valve prosthesis portion in the groove to the ring;
mounting the ring a delivery device;
positioning the delivery device so that the fasteners will engage the target tissue when the fasteners are deployed; and
advancing a shaped plunger member through an interior of the ring to swing said fasteners from a first position to a second tissue engagement position.

19. The method of claim 18 further comprising expanding a support device to an expanded configuration;
moving the support device to engage a bottom surface of the target tissue.

20. The method of claim 18 further comprising removing said delivery device and leaving said ring with the valve prosthetic attached to the target tissue.

21. The method of claim 18 wherein the target tissue is an aortic valve annulus with valve leaflets removed.

22. A kit comprising:
a valve prosthesis delivery device;
a ring having a plurality of fasteners;
a valve prosthesis;
instructions for use setting forth the method of claim 18; and
a container sized to house the valve prosthesis delivery device, the valve prosthesis, and the instructions for use.

23. A kit comprising:
a ring having a plurality of fasteners;
a valve prosthesis;
instructions for use setting forth the method of claim 18; and
a container sized to house the valve prosthesis delivery device, the valve prosthesis, and the instructions for use.
